(19) United States
(12) Patent Application Publication
(10) Pub. No.: US 2012/0245357 A1
(43) Pub. Date: Sep. 27, 2012

(54) ALR2 INHIBITORS AND THEIR SYNTHESIS FROM A NATURAL SOURCE

(75) Inventors: Madhusudana Rao Janaswamy, Hyderabad (IN); Bhanuprakash Reddy Geereddy, Hyderabad (IN); Rama Subba Rao Vidadala, Hyderabad (IN); Muthenna Puppala, Hyderabad (IN)

(73) Assignee: COUNCIL OF SCIENTIFIC & INDUSTRIAL RESEARCH, New Delhi (IN)

(21) Appl. No.: 13/376,661

(22) PCT Filed: Mar. 21, 2011
(86) PCT No.: PCT/IB11/00593

§ 371 Date: Jun. 1, 2012

(30) Foreign Application Priority Data
Mar. 22, 2010 (IN) 682/DEL/2010

(51) Int. Cl. C07D 401/06; C07D 401/14

(52) U.S. Cl. 546/201; 546/277.4

(57) ABSTRACT

A Michael adduct of piplartine, to provide inhibition of ALR2 in vitro (supported by molecular docking) and their potential to suppress the accumulation of sorbitol in erythrocytes when incubated under high glucose conditions; a treatment method using a Michael adduct; and a process for preparing a Michael adduct are provided.

### FIELD OF THE INVENTION

The present invention relates to the identification of new natural agents from (Piper species) Piper chaba, and chemical transformation of piplartine led to the synthesis of novel hybrid compounds as ALR2 inhibitors (ARI). Aldose reductase (AKR1B1 or ALR2; EC: 1.1.1.21) catalyzed accumulation of osmotically active sorbitol which has been implicated in the development of diabetic complications like cataract, retinopathy, neuropathy and nephropathy.

More particularly, the present invention relates to the preparation of few synthetically novel compounds which are synthesized via Michael addition and all adducts inhibited human recombinant ALR2 activity and also suppressed sorbitol accumulation in human RBC under ex vivo high glucose conditions. Thus these compounds might be useful for the treatment and/or prevention of diabetic complications.

### BACKGROUND OF THE INVENTION

According to the latest WHO estimates currently, approximately, 200 million diabetic people are present in the world. This will increase to at least 350 million by the year 2025, which could have a severe impact on human health. Prolonged exposure to uncontrolled chronic hyperglycemia in diabetes can lead to various complications, affecting the cardiovascular, renal, neurological and visual systems. Long-term complications represent the main cause of morbidity and mortality in diabetic patients. Although mechanisms leading to diabetic complications are not completely understood, many biochemical pathways associated with hyperglycemia have been implicated. Among these, the polyol pathway has been extensively studied.

References may be made to Journal “A thirty-year journey in the polyol pathway, Exp. Eye Res. 50 (1990) 567-573” by J. H. Kinoshita wherein it describes that Aldose reductase (ALR2; EC: 1.1.1.21) is the rate-limiting enzyme of the polyol pathway and reduces excess glucose to sorbitol. ALR2 belongs to aldo-keto reductase (AKR) super family, other prominent members of AKR family are aldehyde reductase (ALR1) and small intestine reductase (HSIR or AKR1B10). Though, the actual physiological significance of ALR1 and AKR1B10 are not known, ALR1 is known to play a role in the detoxification of reactive aldehydes.

Under euglycemic conditions, ALR2 plays a minor role in glucose metabolism; however, during diabetes, its contribution is significantly enhanced leading to conversion of excess glucose to sorbitol in insulin independent tissues like nerve, lens, retina and kidney (J. H. Kinoshita, A thirty-year journey in the polyol pathway, Exp. Eye Res. 1990, 50, 567-573, A. Bhatnagar, S. K. Srivastava, Aldose reductase: congenial and injurious profiles of an enigmatic enzyme, Biochem. Med. Metab. Biol. 1992, 48, 91-121). Osmotic stress due to accumulation of high concentrations of sorbitol is postulated to be a major factor in the development of diabetic complications such as neuropathy, nephropathy, retinopathy and cataract.

References may be made to Journals “The pharmacology of aldose reductase inhibitosrs, Annu. Rev. Pharmacol. Toxicol. 1985, 25, 691-714P” by F. Kador, W. G. Robison, J. H. Kinoshita, and “Aldose reductase inhibitors: the end of an era or the need for different trial designs? Diabetes 1997, 46, S82-S89” by M. A. Pfeifer, M. P. Schumer, D. A. Gelber, wherein a number of studies with experimental animals suggest that ALR2 inhibitors (ARI) could be effective in the prevention of certain diabetic complications like cataract, retinopathy, nephropathy and neuropathy. To date, a number of ARI, both synthetic and natural, have been found to improve some diabetic complications in animal experiments and have been developed to the point of clinical evaluation.

References may be made to Journal “Aldose reductase inhibitors and diabetic complications, Am. J. Med., 1987, 83, 298-306” by P. Raskin, J. Rosenstock wherein a wide variety of compounds have been synthesized to inhibit ALR2 and studied in experimental models.

References may be made to Journals “Clinical studies with an aldose reductase inhibitor in the autonomic and somatic neuropathies of diabetes, Metabolism 1986, 35, 83-923” by B. Jaspan, V. L. Towle, R. Maselli, K. Herold, and also “Worldwide pharmacovigilance systems and tolrestat withdrawal, Lancet 1997, 349, 399-400” by M. Foppiano, G. Lombardo, wherein, it reveals that the clinical trials of many ARI have met with limited success, and some of the synthetic ARI were associated with deleterious side effects and poor penetration of target tissues such as nerve and retina. References may be made to Journals “The pharmacology of aldose reductase inhibitors, Annu. Rev. Pharmacol. Toxicol. 25 (1985) 691-714” by P. F. Kador, W. G. Robison, J. H. Kinoshita; “Aldose reductase inhibitors: the end of an era or the need for different trial designs? Diabetes 46 (1997) S82-S89M.A” by Pfeifer, M. P. Schumer, D. A. Gelber; “Aldose reductase inhibitors and diabetic complications, Am. J. Med. 83 (1987) 298-306P” by Raskin, J. Rosenstock wherein largely, two chemical classes of ARI have been tested in phase III trials. While carboxylic acid inhibitors (such as zopolrestat, ponalrestat and tolerestat) have shown poor tissue permeability and are not very potent in vivo, spiroimide (spirohydantoin) inhibitors (like sorbinil) penetrate tissues more efficiently but many have caused skin reactions and liver toxicity. Although strict glycemic control is expected to control or prevent diabetic complications, most individuals with diabetes rarely achieve consistent euglycemia. Hence, agents that can substantially delay or prevent the onset and development of diabetic complications, irrespective of glycemic control, would offer many advantages. In principle, ARI can be included in this category. Thus, intensive research continues to identify and test both synthetic as well as natural products for their therapeutic value to prevent the onset and/or arrest progression of diabetic complications. The plants create unexpected and novel structures to protect themselves from predator organism. By trail and error, several plants and plant products are identified as drugs. Natural product drugs although are highly effective and free from toxic side effects, have a disadvantage with respect to short supply and chemical structure, which makes their manufacture difficult or impossible. Natural product drugs have been a source of lead structure in drug design and development. Semi synthetic analogues or synthetic analogues closely related to the natural product drug of lead are synthesized and screened to disorder their action. In the light of above descriptions, in our isolation work alkanamides, lignans, flavanoids and miscellaneous compounds have been isolated.

References may be made to Journal “Dietary sources of aldose reductase inhibitors: prospects for alleviating diabetic complications, Asia Pac J Clin Nutr. 2008, 17, 558-65 by Saraswat M, Muthenna P, Suryanarayana P, Petrash J M, Reddy G B wherein we have previously reported ARI activity contained in a few spice/dietary sources using in vitro, and ex vivo models, one of them is black Pepper Piper nigrum.

To this continuity we have tested different extracts of P. nigrum, P. longum, and P. chaba, among them extract of P. chaba showed significant activity towards ALR2. The above results encouraged us to do photochemical investigation on P. chaba and led to isolation of 15 bioactive compounds (1-15), which consists of alkamides, lignans, flavanoids and some miscellaneous compounds. All individual compounds were tested against ALR2. Out of the 15 compounds, piplartine and pipernal have shown best ARI activity with IC₅₀ values 160, 310 μM, respectively with human recombinant ALR2. To improve ARI potential further, we have synthesized a series of compounds (20 compounds) by Michael addition with using different substituted indoles as Michael donors and piplartine as Michael acceptor in the presence of iodine as catalyst. All adducts were tested for their ARI activity against ALR2. From these, adducts 3c and 3e has exhibited the highest and similar ARI activity with an IC₅₀ value 4 μM followed by 3d with IC₅₀ value 40 μM, and 2g, 2j with IC₅₀ value 15 and, 8 μM, respectively. Inhibition of recombinant human ALR2 was further substantiated by molecular docking studies wherein the said compounds 3c, 3e and 2j bind to ALR2 making contacts with mentioned residues. In addition to their ARI activity, we have also assessed their potential to suppress the formation of sorbitol in RBC under high glucose conditions in ex vivo system. Hence, we believe that these compounds, 3c, 3e and 2g might be useful for the treatment and/ or prevention of diabetic complications.

### OBJECTIVE OF THE INVENTION

The main object of the present invention is to isolate novel ALR2 inhibitors (ARI) from natural sources and synthesize effective ARI based on the lead molecule piplartine obtained from the natural source.

Another object of the present invention is to measure their bioactivity in terms of ALR2 inhibition and suppression of sorbitol formation under high glucose conditions in ex vivo system.

Yet another object of the present invention is to assign ARI activity to the isolated and synthetic compounds for developing drugs (targeting ARL2) for the treatment and/ or prevention of diabetic complications.

Still another object of the present invention is to synthesize novel synthetic analogues of P. chaba compound, piplartine via Michael addition with substituted indoles, wherein the all adducts obtained in the present invention consists of new synthetic compounds.

Yet another object of the present invention is to further identify ARI activity for these compounds.

Yet another object of the present invention is to further relate to the ARI activity to compounds isolated from P. chaba first time.

### SUMMARY OF THE INVENTION

Accordingly, present invention provides a compound of general formula A

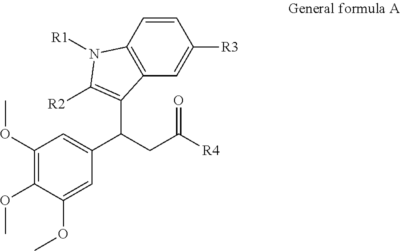

wherein R₁=Hyrogen, Methyl or Benzyl;
   R₂=Methyl or Phenyl;
   R₃=Nitro, Fluro, Bromo, Iodo, Methyl or Methoxy;
   R₄=

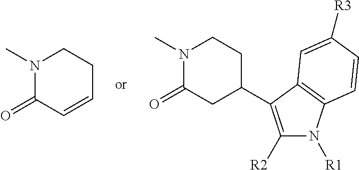

and the representative copounds of general formula A are:

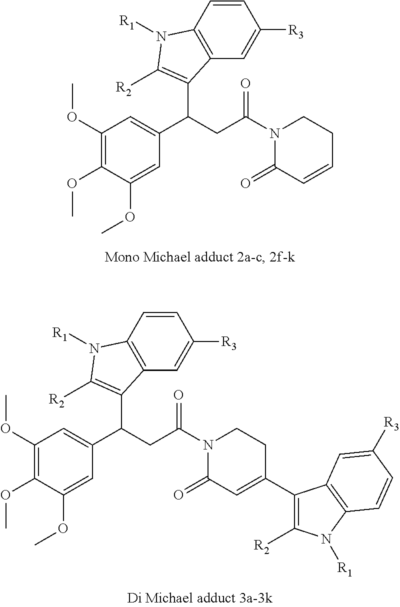

wherein R₁, R₂ and R₃ are the same as defined above.

In an embodiment of the present invention, representative compounds of general formula A comprising:

[1-(3-(1H-indol-3-yl)-3-(3,4,5-trimethoxyphenyl)propanoyl)-5,6-dihydropyridin-2(1H)-one] (2a);

[1-(3-(1H-indol-3-yl)-3-(3,4,5-trimethoxyphenyl)propanoyl)-4-(1H-indol-3-yl)piperidin-2-one] (3a);

[1-(3-(3,4,5-trimethoxyphenyl)-3(1-methyl-1H-indol-3-yl)propanoyl)-5,6-dihydropyridin-2(1H)-one] (2b);

[1-(3-(3,4,5-trimethoxyphenyl)-3-(1-methyl-1H-indol-3-yl)propanoyl)-4-(1-methyl-1H-indol-3-yl)piperidin-2-one] (3b);

[1-(3-(1-benzyl-1H-indol-3-yl)-3-(3,4,5-trimethoxyphenyl)propanoyl)-5,6-dihydropyridin-2(1H)-one] (2c);

[1-(3-(1-benzyl-1H-indol-3-yl)-3-(3,4,5-trimethoxyphenyl)propanoyl)-4-(1-benzyl-1H-indol-3-yl)piperidin-2-one] (3c);

[1-(3-(3,4,5-trimethoxyphenyl)-3-(2-methyl-1H-indol-3-yl)propanoyl)-4-(2-methyl-1H-indol-3-yl)piperidin-2-one] (3d);

[1-(3-(3,4,5-trimethoxyphenyl)-3-(2-phenyl-1H-indol-3-yl)propanoyl)-4-(2-phenyl-1H-indol-3-yl)piperidin-2-one] (3e);

[1-(3-(5-iodo-1H-indol-3-yl)-3-(3,4,5-trimethoxyphenyl)propanoyl)-5,6-dihydropyridin-2(1H)-one] (2f);

[1-(3-(5-iodo-1H-indol-3-yl)-3-(3,4,5-trimethoxyphenyl)propanoyl)-4-(5-iodo-1H-indol-3-yl)piperidin-2-one] (3f);

[1-(3-(5-bromo-1H-indol-3-yl)-3-(3,4,5-trimethoxyphenyl)propanoyl)-5,6-dihydropyridin-2(1H)-one] (2g);

[1-(3-(5-bromo-1H-indol-3-yl)-3-(3,4,5-trimethoxyphenyl)propanoyl)-4-(5-bromo-1H-indol-3-yl)piperidin-2-one] (3g);

[1-(3-(5-fluoro-1H-indol-3-yl)-3-(3,4,5-trimethoxyphenyl)propanoyl)-5,6-dihydropyridin-2(1H)-one] (2h);

[1-(3-(5-fluoro-1H-indol-3-yl)-3-(3,4,5-trimethoxyphenyl)propanoyl)-4-(5-fluoro-1H-indol-3-yl)piperidin-2-one] (3h);

[1-(3-(3,4,5-trimethoxyphenyl)-3-(5-nitro-1H-indol-3-yl)propanoyl)-5,6-dihydropyridin-2(1H)-one] (2i);

[-(3-(3,4,5-trimethoxyphenyl)-3-(5-nitro-1H-indol-3-yl)propanoyl)-4-(5-nitro-1H-indol-3-yl)piperidin-2-one] (3i);

[-(3-(5-methoxy-1H-indol-3-yl)-3-(3,4,5-trimethoxyphenyl)propanoyl)-5,6-dihydropyridin-2(1H)-one] (2j);

[(5-MethoxyIndoleDs)1-(3-(3,4,5-trimethoxyphenyl)-3-(5-methyl-1H-indol-3-yl)propanoyl)-4-(5-methoxy-1H-indol-3-yl)piperidin-2-one] (3j);

[(5-MethylIndoleMs)1-(3-(3,4,5-trimethoxyphenyl)-3-(5-methyl-1H-indol-3-yl)propanoyl)-5,6-dihydropyridin-2(1H)-one] (2k);

[(5-MethylIndoleDs)1-(3-(3,4,5-trimethoxyphenyl)-3-(5-methyl-1H-indol-3-yl)propanoyl)-4-(5-methyl-1H-indol-3-yl)piperidin-2-one] (3k).

In yet another embodiment of the present invention, structural formula of the representative compounds of general formula A comprising:

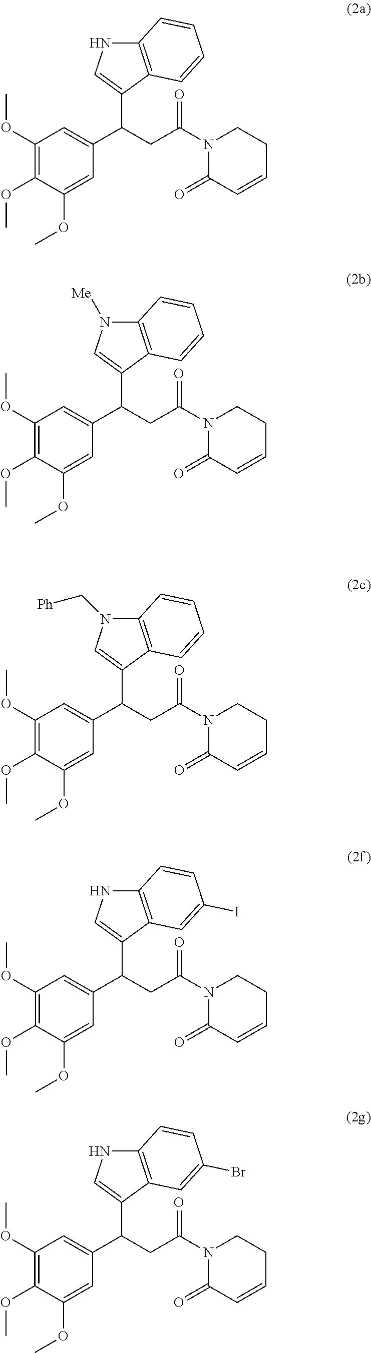

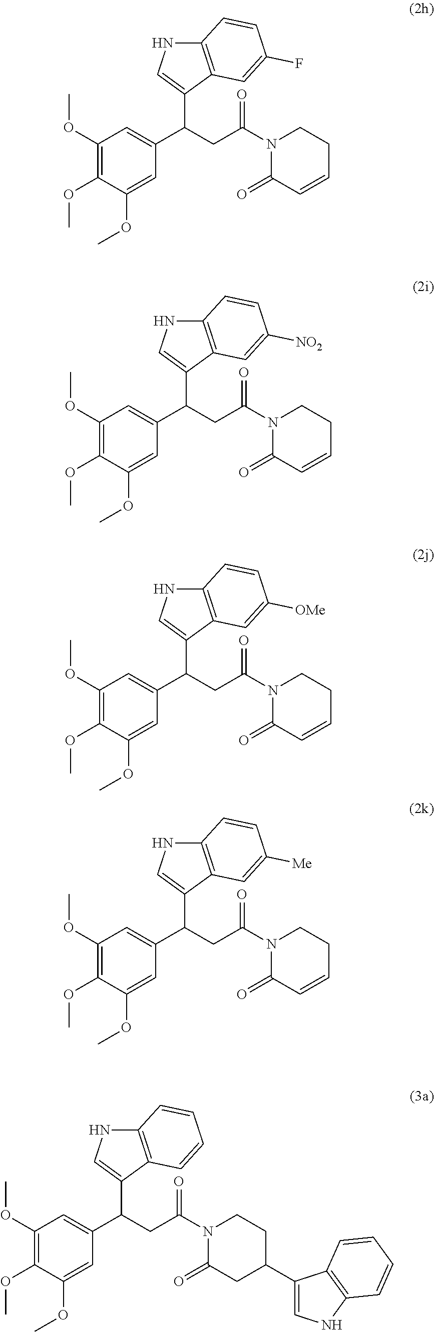

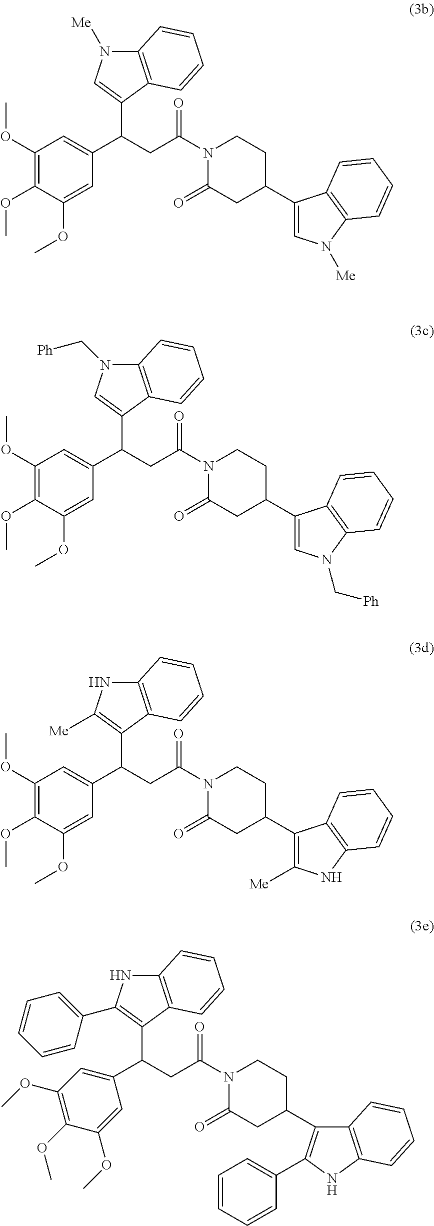

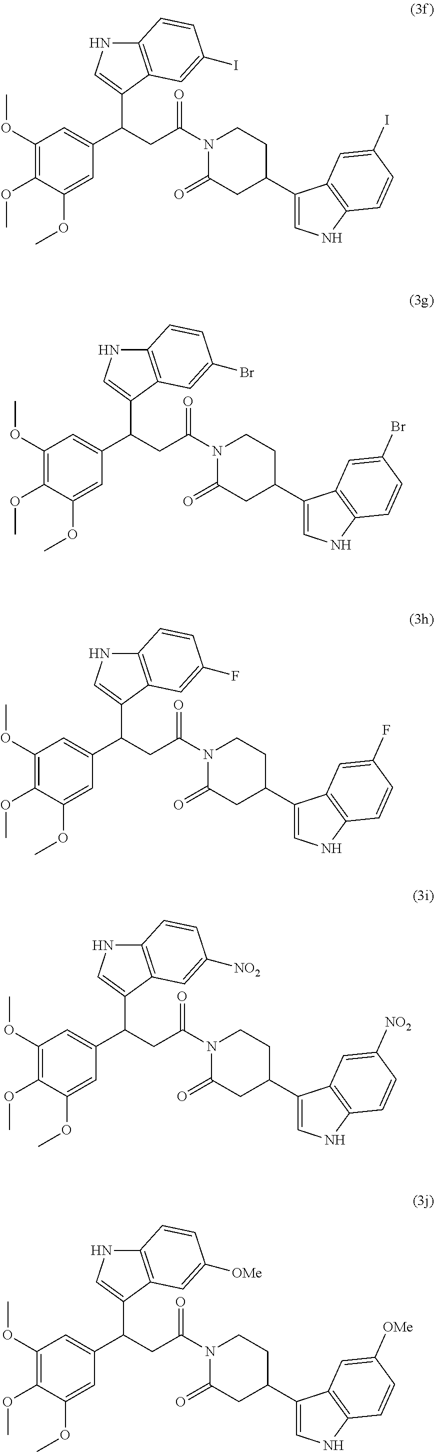

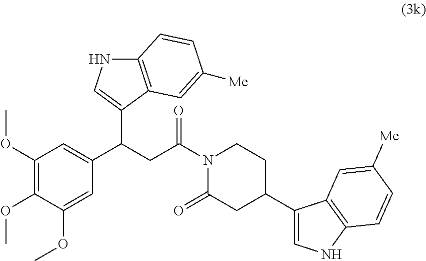

In yet another embodiment of the present invention, compound of general formula A are useful for anti-diabetic complications having (Aldose reductase) ALR2 inhibitory activity.

In yet another embodiment of the present invention, natural compound piplartine is isolated from medicinal plant Piper chaba having (Aldose reductase) ALR2 inhibitory activity and serves as a lead molecule to synthesize novel ALR2 inhibitors.

In yet another embodiment of the present invention, process for the preparation of compound of general formula A by Michael addition and the said process comprising the steps of:
i. mixing piplartine and substituted Indole In the ratio ranging between 1:3 to 1:5 with the catalyst in the ratio ranging between 10 to 12 mole % to obtain a mixture;
ii. refluxing the mixture as obtained in step (i) in solvent at temperature in the range of 60-100° C. for a period in the range of 12 to 48 h till complete conversion;
iii. evaporating the solvent of the refluxed product as obtained in step (ii) followed by washing with saturated hypo solution followed by extraction with chloroform to obtain combined organic layer;
iv. drying the combined organic layer as obtained in step (iii) over anhydrous sodium sulphate followed by evaporating using rotary evaporator to obtain the product;
v. purifying the product as obtained in step (iv) by silica-gel column chromatography to obtain pure product of general formula A.

In yet another embodiment of the present invention, substituted indole used is selected from the group consisting of Indole, 2-methylindole, 1-benzyllindole, 2-methylindole, 2-phenylindole, 5-iodoindole, 5-bromoindole, 5-fluoroindole, 5-nitroindole,5-methoxyindole or 5-methylindole.

In yet another embodiment of the present invention, the catalyst used was selected from the group consisting of Iodine, different Lewis acids and based on selectivity, yield, reaction time preferably iodine is more suitable to synthesize compound general formula A.

In yet another embodiment of the present invention, the solvent used was selected from the group consisting of 1,2-Dichloroethane, Dichloromethane, Methanol and Acetonitrile.

In yet another embodiment of the present invention, compound 3c and 3e exhibiting highest ALR2 inhibition with IC₅₀ values 4 μM.

In yet another embodiment of the present invention, compound 2j and 2g exhibiting ALR2 inhibition with IC₅₀ values 8 and 15 μM respectively.

In yet another embodiment of the present invention, the said compounds are effective in inhibiting human ALR2 in vitro, wherein the said compounds are useful treating the diabetic complications in mammals upon administration of compounds.

In yet another embodiment of the present invention, the said compounds as ALR2 inhibitors is supported by molecular docking data, wherein the said compounds 3c, 3e and 2j bind to ALR2 making contacts with active site residues ALA299, LEU300, SER302.

In yet another embodiment of the present invention, the said compounds are effective in suppressing the formation of sorbitol in RBC under high glucose conditions ex vivo.

In yet another embodiment of the present invention, the said compounds comprise potential against diabetic complications like diabetic cataract, diabetic nephropathy, diabetic neuropathy, diabetic corneal keratopahty, diabetic retinopathy, diabetic dermopathty and other diabetic microangeopathics.

In yet another embodiment of the present invention, the said compounds inhibit epithelial to mesenchymal transition in diabetic retinopathy.

In yet another embodiment of the present invention, the said compounds are used as a prodrug and pharmacological carriers to inhibit diabetic complications like diabetic cataract and diabetic retinopathy.

### BRIEF DESCRIPTION OF THE DRAWINGS

FIG. 1 represents formula of isolated compounds from Piper chaba (1-15)

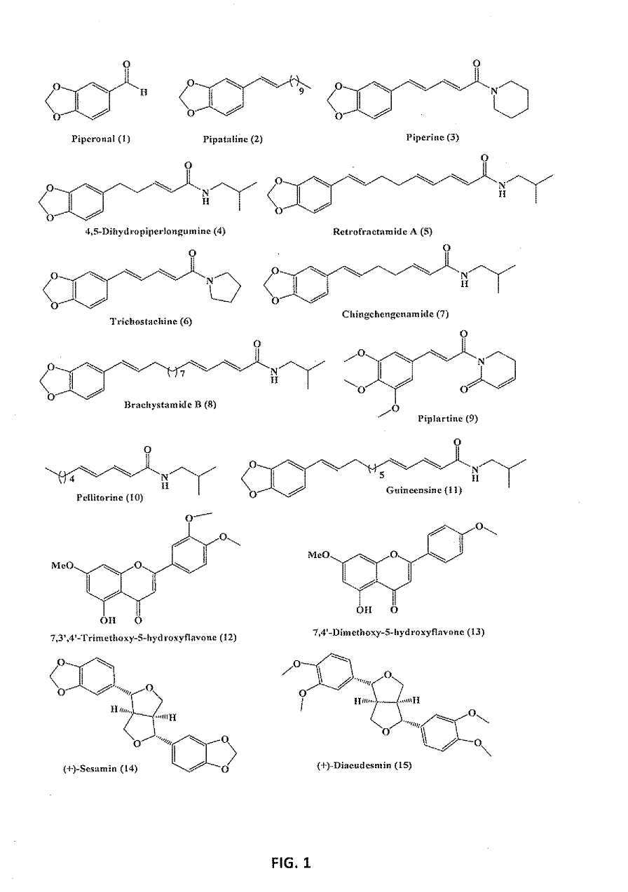

FIG. 2 represent Scheme-1 of Michael addition of piplartine wherein reaction conditions are: a) Indole, I₂ (10 mol %), 1,2-Dichloroethane, 30-120° C., 12-36 h; b) Indole, I₂ (10 mole %), 70° C., Acetonitrile, 12 h; C) Indole, MeOH, 60° C., 12 h

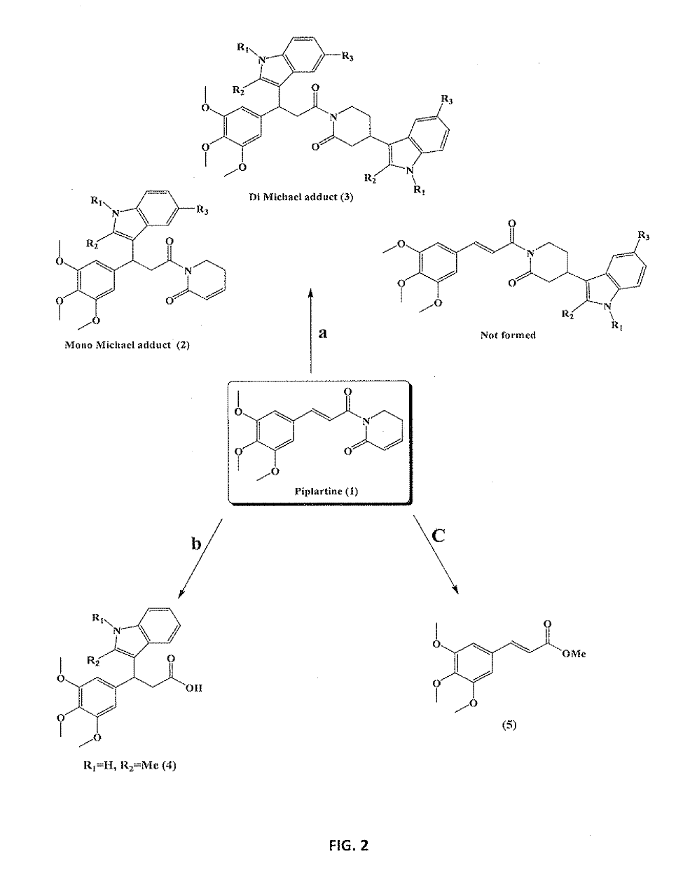

FIG. 3 represent mechanism of Michael addition

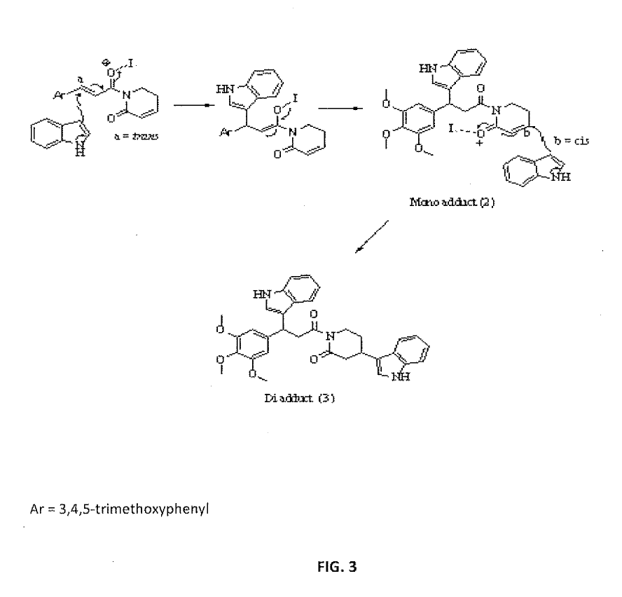

FIG. 4 represent ALR2 inhibition plots for 3c_{;} 3e and 2j against recombinant human ALR2 in vitro. Data presented in Table 4 are average of four experimental values.

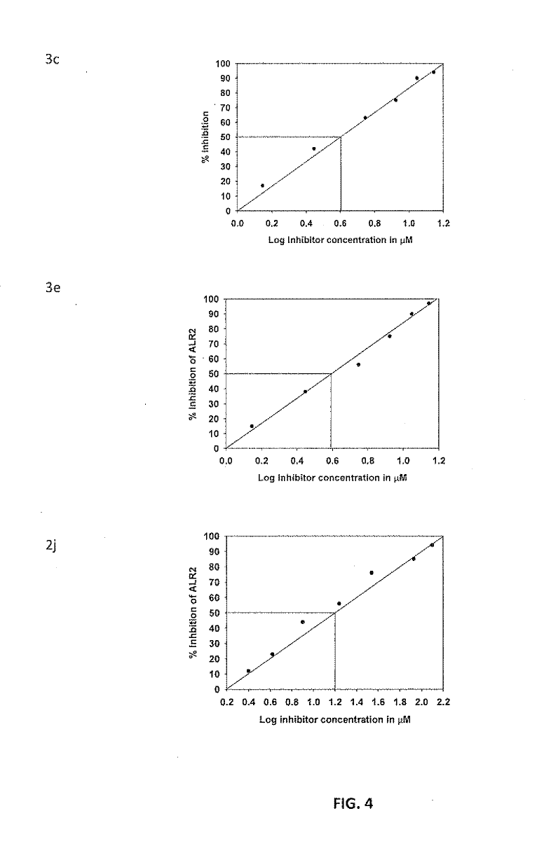

FIG. 5 represent inhibition of sorbitol formation in RBC incubated under high glucose conditions compared to normal conditions for analogues 3c, 3e and 2j. Data represent average of four experiments.

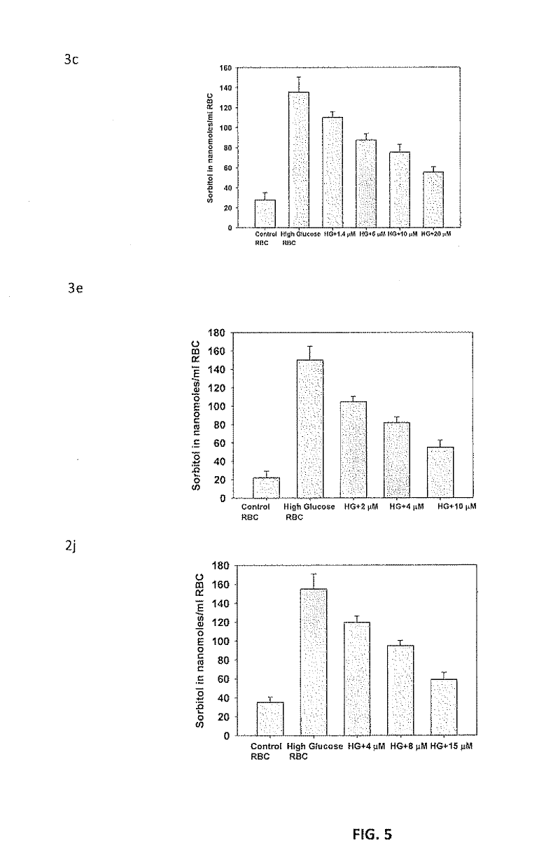

FIG. 6 represent molecular docking studies of 3c, 3e and 2j with ALR2 (PDB: 1PWM).

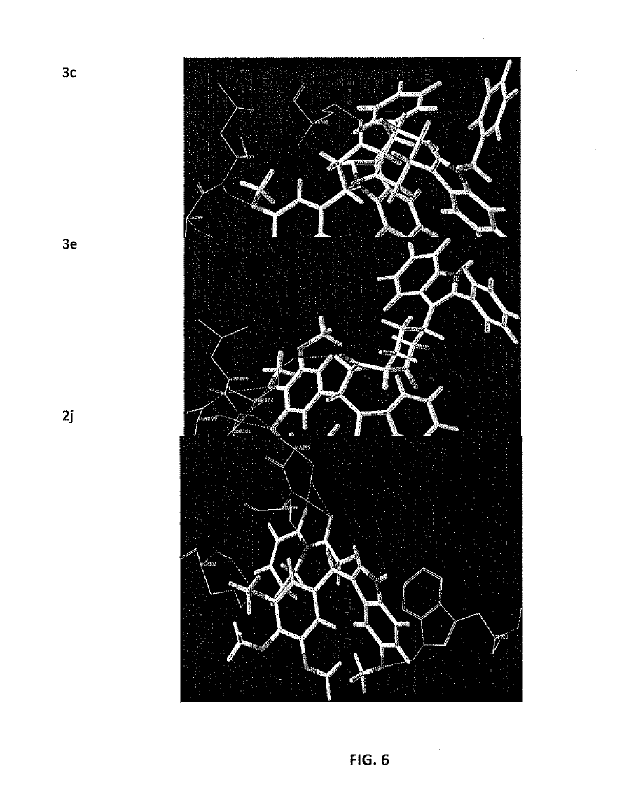

### DETAILED DESCRIPTION OF THE INVENTION

The present invention provides the use of analogs of piplartine with substituted indoles useful for anti-diabetic activity. The said analogs were found to provide inhibition of ALR2 at IC₅₀ value 4 μM for 3c and 3e, and 15, 8 and 40 μM for 2g, 2j and 3d, respectively. In the present invention known ARI quercetin and sorbinil are taken for reference purpose. Quercetin and sorbinil inhibited human recombinant ALR2 at IC₅₀ value 40 and 8 μM level. Thus while all these five molecules are better than quercetin, 3c and 3e are almost on par with sorbinil. Because, among human aldo-keto reductases, ALR2 is unique in its ability to catalyze the NADPH-dependent conversion of glucose to sorbitol (Crosas B, Hyndman D J, Gallego O, Martras S, Pares X, Flynn T G et al. Human aldose reductase and human small intestine aldose reductase are efficient retinal reductases: consequences for retinoid metabolism. Biochem J. 2003; 373: 973-79). Therefore, in addition to their ARI activity, we assessed accumulation of sorbitol in RBC under high glucose conditions (ex vivo) to understand the significance of the said analogues with ARI potential. Further, molecular docking studies were conducted to substantiate the binding pattern and selective inhibition of ALR2 by analogues. It was observed that analogues 3c, 3e and 2j likely interacts with ALR2 at active site residues ALA299, LEU300, SER302 (hydrogen bond distance 1.88, 1.90 and 2.03), ALA299, LEU300, LEU301 and SER302 (hydrogen bond distance 2.4, 1.9, 2.9 and 1.09) and TRP 20, SER302, LEU300, ALA299 (hydrogen bond distance 1.9, 2.7, 2.1 and 1.7) respectively. In vitro incubation of RBC with 55 mM glucose resulted in the accumulation of sorbitol three to four folds higher than the control. Incubation of RBC in the presence of the said analogues under high glucose conditions lead to reduction in the accumulation of intracellular sorbitol. Though, degree of inhibition varied according to the IC₅₀ values of different analogues, on average there was 40-50% reduction with the concentrations equal to their IC₅₀ value of the said analogues. These results indicate the significance of ARI potential of these analogues in terms of preventing the accumulation of intracellular sorbitol. Hence, we believe that the said compounds, particularly, 3c, 3e and 2j might be useful for the treatment and/or prevention of diabetic complications.

Detail Study of Michael Addition on Piplartine

Michael addition reaction is widely recognized as one of the most important carbon-carbon bond forming reactions in organic synthesis. Addition of activated olefins to indoles fulfills the role of synthesis of 3-substituted indoles. Traditionally these compounds have been synthesized by Michael addition (B. M. Trost. On inventing reactions for atom economy. Ace. Chem. Res. 2002, 35, 695) of α,β-unsaturated carbonyl compounds to indoles with strong bases, such as alkali metal alkoxides, hydroxides, amines, and Bronsted acids (S. Zhu, T. Cohen, The preparation of synthetically useful carbonyl-protected α,β-lithio ketones via reductive lithiation. Tetrahedron 1997, 53, 17607; H. Hiemstra, H. Wiberg, Addition of aromatic thiols to conjugated cycloalkenones, catalyzed by chiral beta-hydroxy amines. A mechanistic study of homogeneous catalytic asymmetric synthesis. J. Am. Chem. Soc. 1981, 103, 417; Sulfamic acid-catalyzed Michael addition of indoles and pyrrole to electron-deficient nitroolefins under solvent-free condition. Tetrahedron Lett. 2007, 48, 4297). However, base catalyzed method sometimes suffers from disadvantages of in compatibility with base sensitive functionality and other side reactions such as auto condensation, retro-Michael type decomposition, polymerization, self-condensation, and rearrangements, which in turn decrease the purity and yields of the desired products (L. Novak, P. Kolontis, C. Szantay, D. Aszodi, M. Kajtar, Synthesis and rearrangement of 13-thiaprostanoids. Tetrahedron 1982, 38, 153). In view of current interest in catalytic processes, there is a merit in developing 1,4-addition of activated olefins to indoles using an inexpensive, mild, and nonpolluting reagent. To overcome, these hurdles considerable attention has recently been focused on the use of Lewis acid catalysts including transition metal complex (G. A. Olah, R. Krishnamurty, G. K. S. Prakash. Friedel-Crafts alkylation. In Comprehensive. Organic Synthesis, 1st edn.; Trost, B. M. and I. Fleming, Eds. Pergamon: Oxford, 1991, Vol. III, p. 293. 8; InBr₃: M. Bandini, P. Melchiorre, A. Melloni, A. Umani-Ronchi. A practical indium tribromide catalysed addition of indoles to nitroalkenes in aqueous media. Synthesis 2002, 1110; InCl3: J. S Yadav, S. Abraham, B. V. S. Reddy, G. Sabitha, InCl₃-catalysed conjugate addition of indoles with electron-deficient olefins. Synthesis. 2001, 2165; Yb(OTf)₃ in ScCO₂: I. Komoto, S. Kobayashi, Lewis acid catalysis in supercritical carbon dioxide. Use of poly(-ethylene glycol) derivatives and perfluoroalkylbenzenes as surfactant molecules which enable efficient catalysis in SeCO₂. Org. Chem. 2004, 69, 680; SmI₃: Z.-P. Than, R.-F. Yang, K. Lang, Samarium triiodide-catalyzed conjugate addition of indoles with electron-deficient olefins. Tetrahedron Lett. 2005, 46, 3859, CeCl₃. 7H₂O—NaI—SiO₂: G. Bartoli, M. Bosco, S. Giuli, A. Giuliani, L. Lucarelli, E. Marcantoni, L. Sambri, E. Torregiani, Efficient preparation of 2-Indolyl-1-nitroalkane derivatives employing nitroalkenes as versatile michael acceptors: New practical linear approach to alkyl 9H-b-Carboline-4-carboxylate. J. Org. Chem. 2005, 70, 1941).

However, many of these Lewis acids are highly corrosive, moisture sensitive and require stoichiometric amounts and also provide the products with low diastereoselectivity. This result prompted us to investigate the suitable catalyst to achieve Michael adduct from piplartine. Herein we report iodine acting as an active catalyst for performing Michael addition on α,β-unsaturated amide moieties. Recently, elemental iodine has received considerable attention in organic synthesis because of its high tolerance to air and moisture, low-cost, nontoxic nature and ready availability. The mild Lewis acidity associated with iodine has led to its use in organic synthesis using catalytic to stoichiometric amounts. (H. Togo, S. Iida, Synlett. 2006, 2159-2175, X.-F. Lin, S.-L. Cui, Y.-G. Wang, Tetrahedron Lett. 2006, 47, 4509-4512; (c) W.-Y. Chen,.; J. Lu, Synlett 2005, 1337-1339, L. Royer, S. K. De, R. A. Gibbs, Tetrahedron Lett. 2005, 46, 4595-4597, B. K. Banik, M. Fernandez, C. Alvarez. Tetrahedron Lett. 2005, 46, 2479-2482). To the best of our knowledge this mild. Lewis acid has been used for carbon-carbon bond formation, Iodine-catalyzed Michael addition reaction has been reported on α,β-unsaturated esters, sulphones, nitro olefins (H. Shifan, X. Xuebao, Z. Kexueban. Synthesis of 2,2′-bis(3-hydroxyl-5,5-dimethyl-2-cyclohexen-1-yl) toluene by catalysis with iodine in water under microwave irradiation. Huaiyin Shifan Xueyuan Xuebao Bianjibu, 2008, 7, 239-241; L. S. Jung, L. J. June, K, C-Hyeak, J. Y. Moo, L. B. Min, K. B. Hyo. 2-(N-Hydroxylamino)sulfone synthesis by indium-iodine-triggered aza-Michael type addition of nitroarenes to vinyl sulfones. Tetrahedron Letters 2009, 50, 484-487; L. Chunchi, H. Jianming, M. N. V. Sastry, F. Hulin, T. Zhijay, L. Ju-Tsung, C-Fa, Yao. I₂-catalyzed Michael addition of indole and pyrrole to nitroolefins. Tetrahedron 2005, 61, 11751-11757; C. C-Ming, G. Shijay, M. N. V. Sastry, Y. C-Fa. Iodine-catalyzed Michael addition of mercaptans to α,β-unsaturated ketones under solvent-free conditions. Tetrahedron Letters 2005, 46, 4971-4974; B. Bimal-K. F. Miguel, A. Clarissa. Iodine-catalyzed highly efficient Michael reaction of indoles under solvent-free condition. Tetrahedron Letters 2005, 46, 2479-2482; W. Shun-yi, J. Shun-jun, L. Teck-peng. The Michael addition of indole to α,β-unsaturated ketones catalyzed by iodine at room temperature. Synlett. 2003, 15, 2377-2379).

The reactions of indole with α,β-unsaturated amide moieties like piplartine are not known. Natural product piplartine act as Michael acceptor, it consists of S-trans and S-cis as two α,β-unsaturated amide functionalities. Indole acts as Michael donor. Michael adducts which are formed by this reaction gives 3-substituted indoles, whose synthesis has received attention from organic chemists because they are very important building blocks for biologically active compounds and as prodrugs for use in cancer therapy because oxidants, such as horseradish peroxidase, convert these compounds to products which are toxic to human tumor cells (L. K. Folkes, Wardman, P. Oxidative activation of indole-3-acetic acids to cytotoxic species—a potential new role for plant auxins in cancertherapy. Biochem. Pharr. 2001, 61, 129; O. Greco, S. Rossiter, C. Kanthou, L. K. Folkes, P. Wardman, G. M. Tozer, G. U. Dachs, Horseradish peroxidase-mediated gene therapy: choice of prodrugs in oxic and anoxic tumor conditions. Mol. Cancer Ther. 2001, 1, 151)

To find the optimum conditions towards the catalyst, the Michael addition reaction of piplartine with 3.0 equiv of 5-NO₂ indole was carried out in presence of variety of Lewis and Bronsted acids (Table 2). The highest catalytic activity was attained for the reaction using 10 mol % of Iodine. The role of iodine in this reaction can be attributed to its mild Lewis acid ability, which enhances both the nucleophilicity of indole and electrophilicity of the piplartine via enol forms. The catalytic activitiy of Lewis acids like iodine mainly relies on their coordinating character to assemble both Michael donors and acceptors on their coordination surface. To find the optimum conditions towards the solvent, several reactions were carried out under the solvents like Dichloromethane, 1,2-Dichloroethane, Methanol and Acetonitrile (Table 3) and results were tabulated. Under the solvent acetonitrile adduct formed after Michael reaction, underwent hydrolysis and the product (4) was isolated. In contrast when methanol was used as solvent, methyl ester of 3,4,5-trimethoxycinnamicacid (5) was isolated. Therefore for this meaningly sensitive substrate like piplartine, the use of solvent C₂H₄Cl₂ (DCE) played a vital role for giving Michael adducts. In conclusion, 1,2-Dichloroethane (DCE) is excellent and suitable solvent for this reaction to give target adducts without unwanted products.

The data (Table 1) explained about the reactivity of different substituted indoles with piplartine. Among the 5-substituted indoles the order of reactivity against piplartine was observed as OMe<Me<I═Br<F<NO₂, among the 1-substituted indoles the order of reactivity was observed as Benzyl<Me, among the 2-substituted indoles the order of reactivity was observed as Phenyl<Me. The above data clearly explains that, if electron withdrawing group present at position 5 of indole increases, the reactivity of indole with piplartine increases. If electron donating group present at position 1 of indole increases, the reactivity of indole with piplartine increases. If electron donating group present at position 2 of indole increases, the reactivity of indole with piplartine increases. Among all the Michael donors (substituted indoles) 5-NO₂ indole was highly reactive towards the Michael acceptor (piplartine) and reaction after 3-48 h, 80% of over all products (2i and 3i) were isolated (Table 1, Entry 9). However reaction did not take place with 7-aza indole.

Piplartine it consists of two sites as unsaturated moieties, among them one is trans (path a) and another one is cis (path b) to attract Michael donor to form carbon-carbon single bond. Products as mono adducts (2a-k) were observed via trans, and di adducts (3a-k) were observed via trans and cis, but no mono adduct from cis was observed in entire study. All mono adducts from trans were isolated in good yields except for the entries 4 (compound 2d), 5 (compound 2e) (Table 1) due to electrostatic repulsions between 2-Me indole and 2-Phenyl indole with piplartine. The effect of temperature and volume of solvent also played imperative role for this reaction, in first case study of reaction at 30-80° C. leads to high yield of mono-adduct, and low yield of di-adduct (observed in entry 9, table 1). At 50-100° C. reaction gave both mono and di adducts with more or less equal quantities. At 85-120° C. high yield of di-adduct and low yield of mono-adduct were formed. High volume of solvent leads to formation of mono adduct with high yield, low volume of solvent leads to formation of di adduct with high yield and medium volume of solvent leads to equal quantities of both mono and di-adducts. The enantio, diastereo-selectivity was determined using HPLC. The mono-adduct (2g) formed as racemic mixture with the ratio of 1:1, as analyzed by HPLC (column: Chiral pak IA 250×4.6 mm, 5μ, Flow rate:1.0 ml/min, 225 nm, PDA detector) elution with 15% Isopropanol in Hexane), di-adduct (3e) formed as diastereomers with the ratio of 1:1, as analyzed by HPLC (column: YMC silica 150×4.6 mm, Flow rate: 1.0 ml/min) elution with 4% Isopropanol in Hexane). The ¹H, ¹³C NMR also gave evidence for the occurrence of diastereomers.

In conclusion, we succeed in developing a novel method to effect the Michael addition reaction of indole with natural product piplartine in the presence of iodine as economical mild Lewis-acid catalyst under selective solvent conditions. It should be noted that the method does not require any metal salts and hence it might be of great value as an environmentally welcoming process (T. W. Green, Protecting Groups in Organic Synthesis; Wiley: New York, 1981.) The method offers many advantages for producing several types of Michael adducts with α,β-unsaturated amide moieties containing natural compound like piplartine.

Table: 1 represents different indoles and time, and yield of all adducts

TABLE 1
Michael adducts of different Substituted Indoles, time, product and yield.

| | | | | | Product | | |
|---|---|---|---|---|---|---|---|
| Entry | R₁ | R₂ | R₃ | Time (h) | 2 | 3 | Yield^{a} (2/3) |
| 1 | H | H | H | 36 | 2a | 3a | 20/30 |
| 2 | Me | H | H | 18 | 2b | 3b | 35/35 |
| 3 | Bz | H | H | 20 | 2c | 3c | 30/30 |
| 4 | H | Me | H | 18 | 2d^{b} | 3d | 0/50 |
| 5 | H | Ph | H | 24 | 2e^{b} | 3e | 0/45 |
| 6 | H | H | I | 19 | 2f | 3f | 30/30 |
| 7 | H | H | Br | 17 | 2g | 3g | 30/30 |
| 8 | H | H | F | 15 | 2h | 3h | 35/40 |
| 9 | H | H | NO₂ | 12 | 2i | 3i | 40/40 |
| 10 | H | H | OMe | 22 | 2j | 3j | 20/25 |
| 11 | H | H | Me | 18 | 2k | 3k | 20/30 |

^{a}Isolated yields after column chromatography. All products were characterized by ¹H NMR, ¹³C NMR, IR, HRESIMS spectroscopy.
^{b}Products were not formed due to electrostatic repulsions.

Table: 2 represent optimization of catalyst for Michael addition

TABLE 2
Optimization of catalyst for Michael
addition of indole with piplartine.

| SL. No | Catalyst | Time (h) | Yield (%)^{b} |
|---|---|---|---|
| 1 | CeCl₃•7H₂O (2 equiv) | 48 | 30 (50) |
| 2 | La (NO₂)₃ (2 equiv) | 72 | 15 (70) |
| 3 | NaHSO₄•SiO₂ (2equiv) | 48 | 30 (60) |
| 4 | ZrOCl₂ (2 equiv) | 72 | trace^{c} |
| 5 | La (OTf)₃ (2 equiv) | 72 | 10 (80) |
| 6 | CAN (2 equiv) | 72 | — |
| 7 | Zn Cl₂ (2 equiv) | 72 | trace^{c} |
| 8 | PTSA (2 equiv) | 72 | trace^{c} |
| 9 | Bi (OTf)₃ (2 equiv) | 72 | 10 (80) |
| 10 | Umberlyst-15 (2 equiv) | 72 | 50 (30) |
| 11 | Umberlyst-125 (2 equiv) | 72 | — |
| 12 | Iodine (10 mol %) | 12 | 80 (10) |
| 13 | TBAB (1 equiv) | 72 | trace^{c} |

^{a}All reactions were carried out by using 5-NO₂ indole under solvent DCE at 30-120° C.
^{b}Overall isolated yield of both 2i and 3i adducts after column chromatography, yields in parentheses are recovery of 1.
^{c}Unreacted 1 was mostly recovered.

TABLE 3
Optimization of solvent for Michael addition

| SL. No | Solvent | Yield (%)^{a} |
|---|---|---|
| 1 | Dichloromethane | Trace^{b} |
| 2 | Dichloroethane | 80 |
| 3 | Methanol | —^{c} |
| 4 | Acetonitrile | —^{d} |

^{a}Combined yield of mono and di adducts, all reactions were carried out by using 5-NO₂ indole at 30-120° C.
^{b}Reaction was carried out at room temperature (25 to 30° C.).
^{c}Transesterification product of 3,4,5-trimethoxycinnamicacid methyl ester was isolated.
^{d}Hydrolysis product was isolated.

SAR Studies

All Michael adducts were screened against in-vitro aldose reductase inhibition and IC₅₀ values of selected adducts were summarized in table 4. The Michael adducts obtained by addition of indole to piplartine enhanced the activity by 40 folds. Among all adducts, di adducts showed notable activity than mono adducts. Results are very encouraging when R₁ substituted with benzyl group (3e, IC₅₀=4 μm) rather than methyl and hydrogen groups. Same results were obtained when R₂ substituted with phenyl group (3e, IC₅₀=4 μm), rather than methyl group (3d, IC₅₀=40 μm) also showed good activity. In case of R₃, among all the substitutions methoxy group exhibited considerable activity (2j, IC₅₀=8 μm), among the halogens, bromine showed moderate activity (2g, IC₅₀=60 μm). The above results explained that the adduct needs an active methylene group like benzyl at R₁ position and hydrophobic groups like phenyl at R₂ position and electron donating groups like methoxy at R₃ position are indispensable to show significant activity. However the hydrolysis products 4, 5 were inactive towards the enzyme inhibition.

### EXAMPLES

The following examples are given by way of illustration and therefore should not be construed to limit the scope of the present invention.

### Example 1

Isolation of Piplartine (1)

After collection, the roots were cut into pieces, shade-dried and finally ground to coarse powder. The powdered plant material (1 kg) was extracted with hexane (3 L) in a Soxhlet apparatus for 72 h. The solvent mixture was rota evaporated under reduced pressure to yield a yellowish solid (12 g), which gave the first crop of piplartine (2 g) after crystallization from hexane and dichloromethane (8:2).

Experimental Procedure for (2a & 3a)

To a mixture of piplartine (0.317 g, 1 mmol) and Indole (0.351 g, 3 mmol), Iodine (0.0127 g, 10 mol %) was added. The contents were refluxed in 1,2-dichloroethane (5 ml) for an appropriate time (48 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (4:6) to afford pure product mono adduct (2a) and di adduct (3a).

Experimental Procedure for (2b & 3b)

To a mixture of piplartine (0.317 g, 1 mmol) and 2-methylindole (0.655 g, 5 mmol), Iodine (0.0152 g, 12 mol %) was added. The contents were refluxed in 1,2-dichloroethane (5 ml) for an appropriate time (30 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (3:7) to afford pure product mono adduct (2b) and di adduct (3b).

Experimental Procedure for (2c & 3c)

To a mixture of piplartine (0.317 g, 1 mmol) and 1-benzyllindole (0.621 g, 3 mmol), Iodine (0.0127 g, 10 mol %) was added. The contents were refluxed in 1,2-dichloroethane (5 ml) for an appropriate time (34 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (4:6) to afford pure product mono adduct (2c) and di adduct (3c).

Experimental Procedure for (3d)

To a mixture of piplartine (0.317 g, 1 mmol) and 2-methylindole (0.655 g, 5 mmol), Iodine (0.0152 g, 12 mol %) was added. The contents were refluxed in 1,2-dichloroethane (5 ml) for an appropriate time (36 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (4:6) to afford pure di adduct (3d). Compound 2d (mono adduct) was not observed in this reaction.

Experimental Procedure for (3e)

To a mixture of piplartine (0.317 g, 1 mmol) and 2-phenylindole (0.579 g, 3 mmol), Iodine (0.0152 g, 12 mol %) was added. The contents were refluxed in 1,2-dichloroethane (5 ml) for an appropriate time (30 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (3:7) to afford pure di adduct (3e). Compound 2e (mono adduct) was not observed in this reaction.

Experimental Procedure for (2f & 3f)

To a mixture of piplartine (0.317 g, 1 mmol) and 5-iodoindole (0.729 g, 3 mmol), Iodine (0.0127 g, 10 mol %) was added. The contents were refluxed in 1,2-dichloroethane (5 ml) for an appropriate time (34 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (3:7) to afford pure product mono adduct (2f) and di adduct (3f).

Experimental Procedure for (2g & 3g)

To a mixture of piplartine (0.317 g, 1 mmol) and 5-bromoindole (0.585 g, 3 mmol), Iodine (0.0127 g, 10 mol %) was added. The contents were refluxed in 1,2-dichloroethane (5 ml) for an appropriate time (36 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (3:7) to afford pure product mono adduct (2g) and di adduct (3g).

Experimental Procedure for (2h & 3h)

To a mixture of piplartine (0.317 g, 1 mmol) and 5-fluoroindole (0.405 g, 3 mmol), Iodine (0.0127 g, 10 mol %) was added. The contents were refluxed in 1,2-diehloroethane (5 ml) for an appropriate time (36 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (3:7) to afford pure product mono adduct (2h) and di adduct (3h).

Experimental Procedure for (2i & 3i)

To a mixture of piplartine (0.317 g, 1 mmol) and 5-nitroindole (0.486 g, 3 mmol), Iodine (0.0127 g, 10 mol %) was added. The contents were refluxed in 1,2-dichloroethane (5 ml) for an appropriate time (12 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (4:6) to afford pure product mono adduct (2i) and di adduct (3i).

Experimental Procedure for (2j & 3j)

To a mixture of piplartine (0.317 g, 1 mmol) and 5-methoxyindole (0.441 g, 3 mmol), Iodine (0.0127 g, 10 mol %) was added. The contents were refluxed in 1,2-dichloroethane (5 ml) for an appropriate time (40 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (4:6) to afford pure product mono adduct (2j) and di adduct (3j).

Experimental Procedure for (2k & 3k)

To a mixture of piplartine (0.317 g, 1 mmol) and 5-methylindole (0.393 g, 3 mmol), Iodine (0.0127 g, 10 mol %) was added. The contents were refluxed in 1,2-dichloroethane (5 ml) for an appropriate time (38 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel (60-120 mesh) column chromatography by Ethylacetate:Hexane (4:6) to afford pure product mono adduct (2k) and di adduct (3k).

Experimental Procedure for Compound (4)

To a mixture of piplartine (1 mmol) and Indole (3 mmol), Iodine (10 mol %) was added. The contents were refluxed in methanol (5 ml) for an appropriate time (30 h). The reaction was monitored by thin-layer chromatography (TLC). After complete conversion, the solvent was evaporated, and the product was washed with saturated hypo solution (1.0 ml), and then extracted with chloroform. The combined organic layer was dried over anhydrous sodium sulphate and evaporated using rotary evaporator, purified by silica-gel column chromatography to afford pure product (4).

Experimental Procedure for Compound (5)

To a mixture of piplartine (1 mmol) and 2-Methayl Indole (3 mmol), Iodine (10 mol %) was added. The contents were refluxed in acetonitrile (5 ml) for an appropriate time (30 h). The reaction was monitored by thin-layer chromatography (TLC). After complete conversion, the solvent was evaporated, and the product was washed with saturated hypo solution (10 ml), and then extracted with chloroform. The combined organic layer was dried over anhydrous sodium sulphate and evaporated using rotary evaporator, purified by silica-gel column chromatography to afford pure product (5).

### Example 2

Spectralchemical and Physical Properties of Piplartine, Hydrolysis Products (4, 5), Michael Adducts (2a-2k) and (3a-3k)

5,6-dihydro-1-((E)-3-(3,4,5-trimethoxyphenyl)acryloyl)pyridin-2(1H)-one or Piplartine (1) as white needles; mp. 124° C., IR (KBr) νmax: 1660, 1670 cm⁻¹. ¹H NMR (300 MHz, CDCl₃): δ ppm 2.44-2.52 (2H, m), 3.85 (3H, s), 3.89 (6H, s), 4.04 (2H, t, J=6.61 Hz), 6.03 (1H, td, J=9.6, 1.7 Hz), 6.78 (2H, s), 6.92 (1H, m), 7.41 (1H, d, J=15.48 Hz), 7.64 (1H, d, J=15.48 Hz). ¹³C NMR (75 MHz, CDCl₃): δ 24.3, 41.5, 56.2 (2), 61.0, 105.5 (2), 121.0, 125.5, 130.5, 139.5, 139.9, 143.2, 145.5, 153.5, 165.5, 169.5; HRESIMS m/z 318.1349 [M⁺+H], calcd for C₁₇H₁₉NO₅ 318.1336.

3-(3,4,5-trimethoxyphenyl)-3-(2-methyl-1H-indol-3-yl)propanoic acid (4) as light indigo semi liquid; IR (KBr) νmax: 746, 1125, 1237, 1393, 1458, 1590, 1681, 2934 and 3394 cm⁻¹. ¹H NMR (300 MHz, CDCl₃): δ ppm 2.37 (3H, s), 3.16-3.32 (2H, m), 3.75 (3H, s), 3.81 (3H, s) 4.74 (1H, t, J=7.93 Hz), 6.58 (2H, s), 7.02 (1H, t, J=7.55 Hz), 7.10 (1H, t, J=7.55 Hz), 7.26-7.30 (1H, d, J=7.90 Hz), 7.48-7.52 (1H, d, J=7.90 Hz), 7.82 (1H, br s). ¹³C NMR (75 MHz, CDCl₃): δ 29.7, 37.9, 39.6 55.8, 60.7 (2), 104.6 (2), 110.4, 113.1, 119.0, 119.2, 120.8, 124.9, 134.5, 137.2, 137.5, 139.1, 153.0 (2), 185.5; ESIMS m/z C₂₁ H₂₃ N O₅ [M⁺+Cl]⁻ 369.0.

(E)-methyl 3-(3,4,5-trimethoxyphenyl)acrylate (5): as white solid; ¹H NMR (300 MHz, CDCl₃): δ ppm 3.80 (3H, s), 3.86 (3H, s), 3.89 (6H, s), 6.30 (1H, d, J=15.86 Hz), 6.72 (2H, s), 7.57 (1H, d, J=15.86 Hz). ¹³C NMR (75 MHz, CDCl₃): δ 51.7, 56.4 (2), 60.6, 105.6 (2), 116.9, 129.0, 144.9, 153.8 (2), 167.0; ESIMS m/z C₁₃H₁₆O₅ [M⁺+H] 253.0

1-(3-(1H-indol-3-yl)-3-(3,4,5-trimethoxyphenyl)propanoyl)-5,6-dihydropyridin-2(1H)-one (2a): as a pale yellow semi liquid; IR (KBr) νmax: 812, 866, 929, 1037, 1157, 1200, 1248, 1344, 1444, 1496, 1536, 1610, 1653, 1740, 2857, 2921 and 3414 cm⁻¹. ¹H NMR (300 MHz, CDCl₃): δ ppm 2.16 (2H, m), 3.74 (2H, m), 3.78 (9H, s), 3.84-3.94 (2H, m), 4.85 (1H, t, J=7.5 Hz), 5.95 (1H, td, J=9.6, 1.7 Hz), 6.59 (2H, s), 6.79-6.87 (1H, m), 7.02-7.09 (2H, m), 7.12-7.20 (1H, m), 7.33 (1H, d, J=8.1 Hz), 7.55 (1H, d, J=7.9 Hz), 8.12 (NH, br s). ¹³C NMR (100 MHz, CDCl₃): δ 24.5, 39.6, 41.2, 44.5, 56.0 (2), 60.6, 104.7 (2), 111.0, 118.9, 119.5 (2), 121.3, 122.0 (2), 125.6, 126.6, 136.4, 139.9, 145.4 (2), 152.9, 165.9, 175.0; HRESIMS m/z 457.1745 [M⁺+Na], calcd for C₂₅H₂₆N₂O₅ 457.1734.

1-(3-(1H-indol-3-yl)-3-(3,4,5-trimethoxyphenyl)propanoyl)-4-(1H-indol-3-yl)piperidin-2-one (3a): as a pale yellow semi liquid; IR (KBr) νmax: 588, 663, 747, 818, 909, 1005, 1124, 1175, 1235, 1333, 1389, 1422, 1458, 1504, 1591, 1687, 2362, 2852, 2923 and 3361 cm⁻¹. ¹H NMR (300 MHz, CDCl₃): δ ppm 1.73-2.09 (2H, m), 2.14 (1H, m), 2.58-2.75 (1H, m), 2.86-2.97 (1H, dd, J=17.1, 5.6, Hz), 3.34-3.69 (2H, m), 3.76 (3H, s), 3.79 (6H, s), 3.85-4.03 (2H, m), 4.85 (1H, t, J=7.7 Hz), 6.59-6.62 (2H, s), 6.69 (1H, d, J=2.0, Hz), 6.85 (1H, d, J=1.8 Hz), 7.04-7.14 (2H, m), 7.14-7.23 (2H, m), 7.36 (2H, m), 7.55 (2H, m), 8.02-8.13 (2H, NH, br t, J=8.8 Hz). ¹³C NMR (75 MHz, CDCl₃): δ 28.6, 30.0, 39.2, 40.8, 43.9, 44.7, 55.9 (2), 60.9, 105.2 (2), 110.4, 110.6, 112.0, 112.2, 118.6, 118.9, 119.1 (2), 119.3, 120.8 (2), 126.3, 127.5, 130.5, 132.1, 135.2, 135.3, 139.9, 152.8 (2), 173.4, 176.2; HRESIMS m/z 574.2320 [M⁺+Na], calcd for C₃₃H₃₃N₃O₅ 574.2312.

1-(3-(3,4,5-trimethoxyphenyl)-3-(1-methyl-1H-indol-3-yl)propanoyl)-5,6-dihydropyridin-2(1H)-one (2b): as a pale yellow semi liquid; IR (KBr) νmax: 770, 1001, 1125, 1188, 1278, 1317, 1460, 1504, 1646, 2852 and 2923 cm⁻¹. ¹H NMR (300 MHz, CDCl₃): δ ppm 2.10-2.23 (2H, m), 3.65-3.73 (2H, m), 3.74 (3H, s), 3.78 (3H, s), 3.80 (6H, s), 3.80-3.94 (2H, m), 4.83 (1H, t, J=7.3 Hz), 5.92-5.98 (1H, td, J=9.8, 1.5 Hz), 6.59 (2H, s), 6.78-6.87 (1H, m), 6.88 (1H, br s) 7.02-7.08 (1H, m), 7.26 (1H, d, J=8.2 Hz), 7.53-7.57 (1H, d, J=8.1 Hz), 7.15-7.22 (1H, m). ¹³C NMR (75 MHz, CDCl₃): δ 24.5, 29.6, 32.6, 41.2, 45.0, 56.0 (2), 60.7, 104.9 (2), 109.0, 117.5, 118.8 (2), 119.4, 121.6, 125.5 (2), 126.1, 127.0, 136.2, 137.1, 139.9, 145.4, 152.4 (2), 165.5, 174.9. HRESIMS m/z 471.1901 [M⁺+Na], calcd for C₂₆H₂₈N₂O₅ 471.1890.

1-(3-(3,4,5-trimethoxyphenyl)-3-(1-methyl-1H-indol-3-yl)propanoyl)-4-(1-methyl-1H-indol-3-yl)piperidin-2-one (3b): as a pale yellow semi liquid; IR (KBr) νmax: 665, 743, 819, 1011, 1126, 1175, 1233, 1326, 1375, 1422, 1464, 1505, 1590, 1689, 2852, and 2927 cm⁻¹. ¹H NMR (300 MHz, CDCl₃): δ ppm 1.67-1.88 (1H, m), 1.97-2.10 (1H, m), 2.52-2.71 (1H, m), 2.74-2.96 (1H, m), 2.23-3.58 (2H, m), 3.63-3.97 (18H, m), 4.77 (1H, m), 6.52-6.63 (2H, m), 6.66-6.80 (1H, m), 6.82-6.95 (1H, m), 6.97-7.11 (2H, m), 7.13-7.33 (4H, m), 7.40-7.58 (2H, m). ¹³C NMR (75 MHz, CDCl₃): δ 29.7, 29.4, 30.0, 32.7, 40.1, 41.4, 43.4, 45.5, 56.2 (2), 60.2, 105.6 (2), 109.0, 109.0, 116.6, 117.6, 118.7, 118.9, 119.0 (2), 119.1, 119.7, 121.7 (2), 122.1, 124.6, 126.1, 126.5, 126.5, 127.2, 136.5, 137.2, 139.9, 153.0 (2), 172.2, 175.5. HRESIMS m/z 602.2617 [M⁺+Na], calcd for C₃₅H₃₇N₃O₅ 602.2625.

1-(3-(1-benzyl-1H-indol-3yl)-3-(3,4,5-trimethoxyphenyl)propanoyl)-5,6-dihydropyridin-2(1H)-one (2c): as a pale yellow semi liquid; IR (KBr) νmax: 819, 1124, 1460, 1638, 2055, 2362, 2851, 2922, 1444, 1496, 1536, 1610, 1653, 1740, 2857, 2921 and 3414 cm⁻¹. ¹H NMR (300 MHz, CDCl₃): δ ppm 2.06-2.20 (2H, m), 3.64-3.74 (2H, m), 3.77 (6H, s), 3.78 (3H, s), 3.81-3.93 (2H, m), 4.86 (1H, t, J=7.5 Hz), 5.29 (2H, s), 5.90-5.96 (1H, td, J=9.6, 1.7 Hz), 6.57 (2H, s), 6.76-6.84 (1H, m), 6.99-7.09 (4H, m), 7.10-7.16 (1H, m), 7.19-7.31(H, m), 7.50-7.57 (1H, d, J=7.9 Hz). ¹³C NMR (75 MHz, CDCl₃): δ 29.6, 41.3, 45.3, 49.9, 56.0 (2), 60.0, 104.9 (2), 109.6, 118.2, 119.1, 119.6, 121.9, 125.4, 125.6, 126.5 (2), 127.4, 127.5, 128.6 (2), 136.2, 136.8, 137.7, 139.9, 145.3 (2), 152.9 (2), 165.5, 174.9. HRESIMS m/z 547.2225 [M⁺+Na], card for C₃₂H₃₂N₂O₅ 547.2203.

1-(3-(1-benzyl-1H-indol-3-yl)-3-(3,4,5-trimethoxyphenyl)propanoyl)-4-(1-benzyl-1H-indol-3-yl)piperidin-2-one (3c): as a pale yellow semi liquid; IR (KBr) νmax: 744, 1008, 1124, 1176, 1236, 1328, 1460, 1502, 1640, 2063, 2924 cm⁻¹. ¹H NMR (300 MHz, CDCl₃): δ ppm 1.64-1.81 (1H, m), 1.96-2.18 (1H, m), 2.48-2.65 (1H, m), 2.80-2.91 (1H, in), 3.231-3.49 (2H, m), 3.58-3.71 (2H, m), 3.75 (10H, br s), 4.79 (1H, t, J=7.7 Hz), 5.21 (2H, s), 5.29 (2H, s), 6.54 (2H, brs), 6.93-7.13 (9H, m), 7.13-7.31 (9H, m), 7.44-7.52 (2H, d, J=7.5 Hz). ¹³C NMR (75 MHz, CDCl₃): δ 29.4, 29.8, 30.2, 39.9, 41.6, 43.7, 45.7, 50.0, 56.0 (2), 60.0, 105.0 (2), 109.6, 110.0, 117.5, 118.1, 118.9, 119.4 (2), 119.8 (2), 122.1, 122.3, 124.0, 125.5, 126.5 (2), 126.7 (2), 127.6, 127.7 (2), 128.8 (2), 136.5, 136.9, 137.4, 137.8, 139,4, 153.0 (2), 172.8, 175.6. HRESIMS m/z 732.3435 [M⁺+H], calcd for C₄₇H₄₅N₃O₅ 732.3432.

1-(3-(3,4,5-trimethoxyphenyl)-3-(2-methyl-1H-indol-3-yl)propanoyl)-4-(2-methyl-1H-indol-3-yl)piperidin-2-one (3d)): as a pale yellow semi liquid; IR (KBr) νmax: 600, 674, 744, 838, 921, 1006, 1125, 1175, 1244, 1330, 1424, 1459, 1505, 1590, 1691, 2361, 2932, 3396 and 3738 cm⁻¹. ¹H NMR (300 MHz, CDCl₃): δ ppm 1.66-1.86 (2H, m), 2.26 (3H, d, J=6.4 Hz), 2.41 (3H, d, J=3.5 Hz), 2.46-2.66 (1H, m), 2.83-3.17 (1H, m), 3.18-3.57 (2H, m), 3.73 (6H, s), 3.79 (3H, s), 3.81-3.91 (1H, m), 4.01-4.21 (1H, m), 4.89 (1H, m), 6.566 (2H, s), 6.99-7.15 (4H, m), 7.18-7.33 (3H, m), 7.62 (1H, m), 7.99 (NH, br s), 8.06 (NH, br s). ¹³C NMR (75 MHz, CDCl₃): δ 28.5, 30.0, 31.2, 38.6, 39.2, 40.6, 43.9, 44.7, 55.9(2), 60.6, 104.7 (2), 110.4, 110.5, 112.0, 112.5, 118.5, 119.1, 119.2, 119.3, 120.6, 126.4, 127.5, 130.5, 132.2, 135.2, 135.3, 136.0, 140.0, 140.1, 152.8 (2), 173.4, 176.1. HRESIMS m/z 602.2620 [M⁺+Na], calcd for C₃₅H₃₇N₃O₅ 602.2625.

1-(3-(3,4,5-trimethoxyphenyl)-3-(2-phenyl-1H-indol-3-yl)propanoyl)-4-(2-phenyl-1H-indol-3-yl)piperidin-2-one (3e): as a pale yellow semi liquid; IR (KBr) νmax: 861, 609, 666, 700, 744, 770, 836, 921, 1006, 1125, 1175, 1242, 1320, 1422, 1455, 1501, 1592, 1687, 2845, 2928, 3056 and 3394 cm⁻¹. ¹H NMR (300 MHz, CDCl₃): δ ppm 1.50-1.74 (1H, m), 1.78-1.96 (1H, m), 2.30-2.61 (1H, m), 2.77-3.04 (1H, m), 3.05-3.34 (2H, m), 3.43-3.65 (1H, m), 3.69 (6H, s), 3.79 (3H, s), 3.85-4.28 (2H, m), 5.04 (1H, m), 6.61 (1H, s), 6.62 (1H, s), 7.01-7.12 (1H, m), 7.12-7.23 (2H, m), 7.27-7.34 (1H, m), 7.34-7.49 (10H, m), 7.50-7.58 (2H, d, J=7.9 Hz), 7.68-7.81 (1H, m), 7.97 (1H, s), 8.10 (NH, br s), 8.11 (NH, br s). ¹³C NMR (75 MHz, CDCl₃): δ 29.0, 29.6, 31.3, 37.6, 40.8, 44.4, 55.9 (2), 60.7, 104.7 (2), 105.9, 106.6, 110.9, 111.1, 111.3, 112.0, 113.2, 113.4, 119.6, 119.8, 120.5, 121.3, 122.0, 122.2, 124.9, 125.9, 126.3, 128.0, 128.1, 128.2, 128.7, 128.8, 130.3, 131.7, 132.7, 134.9, 136.1, 137.6, 140.1, 152.9 (2), 160.1, 173.2. HRESIMS m/z 742.2685 [M⁺+K], calcd for C₄₅H₄₁N₃O₅ 742.2678.

1-(3-(5-iodo-1H-indol-3-yl)-3-(3,4,5-trimethoxyphenyl)propanoyl)-5,6-dihydropyridin-2(1H)-one (2f); as a pale yellow semi liquid; IR (KBr) νmax: 590, 656, 784, 814, 878, 909, 1000, 1133, 1179, 1231, 1301, 1328, 1360, 1387, 1458, 1504, 1588, 1684, 2361, 2828, 2926, 2994, 3055, 3400 cm⁻¹. ¹H NMR (300 MHz, CDCl₃): δ ppm 2.16-2.25 (2H, m), 3.61-3.79 (2H, m), 3.80 (9H, s), 3.81-3.95 (2H, m), 4.78 (1H, t, J=7.7 Hz), 5.95-6.01 (1H, td, J=9.8, 1.7 Hz), 6.56 (2H, s), 6.82-6.90 (1H, m), 7.02 (1H, br d, J =2.2 Hz), 7.09-7.14 (1H, d, J=8.4 Hz), 7.38-7.43 (1H, dd, J=8.4, 1.7 Hz), 7.87 (1H, d, J=1.3 Hz), 8.09 (NH, br s). ¹³C NMR (100 MHz, CDCl₃): δ 24.7, 39.4, 41.4, 45.1, 55.9 (2), 61.0, 105.0 (2), 113.2, 118.3, 122.2, 125.7, 128.1, 129.2, 130.3, 130.5, 135.5, 136.2, 139.4, 145.4, 152.8 (2), 165.5, 174.8. HRESIMS m/z 599.0456 [M⁺+K], calcd for C₂₅H₂₅IN₂O₅ 599.0440.

1-(3-(5-iodo-1H-indol-3-yl)-3-(3,4,5-trimethoxyphenyl)propanoyl)-4-(5-iodo-1H-indol-3-yl)piperidin-2-one (3f): as a pale yellow semi liquid; IR (KBr) νmax: 664, 770, 877, 1002, 1124, 1175, 1222, 1307, 1387, 1421, 1458, 1505, 1591, 1686, 2361, 2851, 2923 and 3419 cm⁻¹. ¹H NMR (300 MHz, CDCl₃): δ ppm 1.75-1.90 (1H, m), 2.08-2.20(1H, m), 2.58-2.74 (1H, m), 2.88-3.00 (1H, dd, J=17.3, 5.8, Hz), 3.30-3.40 (1H, m), 3.48-3.76 (3H, m), 3.82 (6H, s), 3.83 (3H, s), 3.84-3.98 (1H, m), 4.77 (1H, t, J=7.5 Hz), 6.55 (1H, s), 6.57 (1H, s), 6.64 (1H, d, J=1.7, Hz), 7.03 (1H, d, J=2.0 Hz), 7.11-7.19 (2H, m), 7.39-7.49 (2H, m), 7.83-7.90 (2H, m), 8.10 (NH, br s), 8.18 (NH, br s). ¹³C NMR (75 MHz, CDCl₃): δ 29.5, 30.7, 39.6, 43.1, 45.4, 49.4, 56.1 (2), 60.9, 104.9 (2), 113.1, 113.3, 117.6, 118.2, 121.0, 122.3, 127.7, 128.3, 129.2, 130.4, 130.7, 135.5, 139.3, 139.6, 143.8, 144.3, 145.3, 145.8, 153.1 (2), 173.0, 175.4. HRESIMS m/z 826.0244 [M⁺+Na], calcd for C₃₃H₃₁I₂N₃O₅ 826.0245.

1-(3-(5-bromo-1H-indol-3-yl)-3-(3,4,5-trimethoxyphenyl)propanoyl)-5,6-dihydropyridin-2(1H)-one (2g): as a pale yellow semi liquid; IR (KBr) νmax: 667, 770, 884, 1000, 1124, 1176, 1223, 1323, 1422, 1460, 1505, 1592, 1687, 2852, 2923, and 3430 cm⁻¹. ¹H NMR (300 MHz, CDCl₃): δ ppm 2.18 (2H, m), 3.61-3.75 (2H, m), 3.79 (6H, s), 3.81 (3H, s), 3.84-3.94 (2H, m), 4.78 (1H, t, J=7.5 Hz), 5.94-6.00 (1H, td, J=9.8, 1.7 Hz), 6.56 (2H, s), 6.80-6.89 (1H, m), 7.07 (1H, d, J=2.0 Hz), 7.02-7.23 (2H, m), 7.65 (1H, br d, J=1.5 Hz), 8.10 (NH, br s). ¹³C NMR (75 MHz, CDCl₃): δ 24.5, 39.2, 41.3, 45.2, 56.2 (2), 60.7, 105.0 (2), 112.5, 112.7, 118.8, 121.9, 122.6, 125.0, 125.2, 125.6, 128.4, 135.0, 139.3, 145.5, 153.0 (2), 165.6, 174.6. HRESIMS m/z 535.0839 [M⁺+Na], calcd for C₂₅H₂₅BrN₂O₅ 535.0859.

1-(3-(5-bromo-1H-indol-3-yl)-3-(3,4,5-trimethoxyphenyl)propanoyl)-4-(5-bromo-1H-indol-3-yl)piperidin-2-one (3g): as a pale yellow semi liquid; IR (KBr) νmax: 593, 636, 671, 794, 858, 882, 926, 1000, 1048, 1122, 1176, 1241, 1292, 1322, 1363, 1422, 1458, 1506, 1592, 1669, 1700, 2361, 2929, 3355 and 3737 cm⁻¹. ¹H NMR (300 MHz, CDCl₃): δ ppm 1.74-1.89 (1H, m), 2.07-2.22 (1H, m), 2.59-2.74 (1H, m), 2.83-2.98 (1H,), 3.27-3.40 (1H, m), 3.54-3.73 (1H, m), 3.77 (6H, s), 3.80 (3H, s), 3.82-3.98 (2H, m), 4.78 (1H, t, J=7.3 Hz), 6.56 (2H, s), 6.68 (1H, d, J=2.2 Hz), 6.91 (1H, d, J=2.4 Hz), 7.19-7.30 (5H, m), 7.62-7,69 (2H, m), 8.19 (NH, br s), 8.24 (NH, br s). ¹³C NMR (75 MHz, CDCl₃): δ 29.6, 31.5, 39.6, 41.1, 42.9, 45.4, 56.1 (2), 60.8, 105.0 (2), 112.7, 112,8, 117.4, 117.8, 118.5, 118.6, 121.1, 121.4, 121.9, 122.5, 122.7, 125.0, 125.2, 125.3, 127.7, 128.4, 135.0, 139,9, 153.0 (2), 173.0, 175.4. HRESIMS m/z 732.0524 [M⁺+Na], calcd for C₃₃H₃₁Br₂N₃O₅ 732.0523.

1-(3-(5-fluoro-1H-indol-3-yl)-3-(3,4,5-trimethoxyphenyl)propanoyl)-5,6-dihydropyridin-2(1H)-one (2h): as a pale yellow semi liquid; IR (KBr) νmax: 721, 772, 820, 1004, 1125, 1177, 1220, 1305, 1383, 1462, 1587, 1690, 2852, 2923 and 3367 cm⁻¹. ¹H NMR (300 MHz, CDCl₃): δ ppm 2.14-2.26 (2H, m), 3.64-3.76 (2H, m), 3.79 (6H, s), 3.80 (3H, s), 3.81-3.91 (2H, m), 4.77 (1H, t, J=7.3 Hz), 5.93-5.99 (1H, td, J=9.6, 1.7 Hz), 6.55 (2H, s), 6.80-6.95 (2H, m), 7.12 (1H, d, J=2.0 Hz), 7.13-7.18 (1H, m), 7.20-7.25 (1H, m), 8.05 (NH, br s). ¹³C NMR (75 MHz, CDCl₃): δ 24.5, 39.6, 41.2, 45.0, 56.2 (2), 60.7, 104.2, 105.0 (2), 110.3, 110.7, 111.5, 111.7, 119.2, 119.3, 123.1, 125.6, 132.9, 139,5, 145.5, 153.0 (2), 165.6, 174.6. HRESIMS m/z 491.1380 [M⁺+K], calcd for C₂₅H₂₅FN₂O₅ 599.1379.

1-(3-(5-fluoro-1H-indol-3-yl)-3-(3,4,5-trimethoxyphenyl)propanoyl)-4-(5-fluoro-1H-indol-3-yl)piperidin-2-one (3h): as a pale yellow semi liquid; IR (KBr) νmax: 812, 866, 929, 1037, 1157, 1200, 1248, 1344, 1444, 1496, 1536, 1610, 1653, 1740, 2857, 2921 and 3414 cm⁻¹. ¹H NMR (300 MHz, CDCl₃): δ ppm 1.76-1.93 (1H, m), 2.09-2.21 (1H, m), 2.60-2.74 (1H, m), 2.83-2.99 (1H, m), 3.27-3.42 (1H, m), 3.54-3.75 (2H, m), 3.80 (6H, s), 3.81 (3H, s), 3.83-3.98 (1H, m), 4.77 (1H, t, J=7.1.7 Hz), 6.56 (1H, s), 6.57 (1H, s), 6.88-7.01(2H, in), 7.10-7.16 (1H, m), 7.16-7.22 (2H, m), 7.23-7.25 (1H, m), 7.27-7.32 (2H, m), 8.11 (NH, br d J=7.5 Hz), 8.18 (NH, br s). ¹³C NMR (75 MHz, CDCl₃): δ 28.5, 29.5, 39.8, 41.2, 42.9, 45.3, 56.2 (2), 60.7, 103.5, 105.0 (2), 110.3, 110.7, 110.9, 111.7, 112.1, 117.8, 118.1, 119.0, 121.8, 123.1, 126.3, 127.1, 133.0, 136.3, 139.5, 153.0 (2), 156.1, 159.2, 173.1, 175.5. HRESIMS m/z 610.2127 [M⁺+Na], calcd for C₃₃H₃₁F₂N₃O₅ 610.2124.

1-(3-(3,4,5-trimethoxyphenyl)-3-(5-nitro-1H-indol-3-yl)propanoyl)-5,6-dihydropyridin-2(1H)-one (2i): as a pale yellow semi liquid; IR (KBr) νmax: 605, 657, 741, 775, 819, 899, 1015, 1125, 1180, 1232, 1331, 1385, 1424, 1463, 1514, 1589, 1623, 1684, 2358, 2837, 2933, and 3366 cm⁻¹. ¹H NMR (300 MHz, CDCl₃): δ ppm 2.22-2.33 (2H, m), 3.67-3.78 (2H, m), 3.80 (3H, s), 3.82 (6H, s), 3.84-3.98 (2H, m), 4.89 t, J=7.3 Hz), 5.96-6.02 (1H, td, J=9.8, 1.5 Hz), 6.58 (2H, s), 6.85-6.95 (1H, m), 7.24 (1H, brd, J=1.8 Hz), 7.36 (1H, d, J=9.0 Hz), 8.05-8.10 (1H, dd, J=9.0, 2.0 Hz), 8.50 (NH, br s), 8.52 (1H, br d, J=2.0 Hz). ¹³C NMR (75 MHz, CDCl₃): δ 24.5, 39.2, 41.1, 45.9, 56.2 (2), 60.7, 104.7 (2), 111.1, 116.6, 117.8, 121.7, 124.4, 125.6, 126.1, 136.5, 139.1, 139.5, 141.5, 145.7, 153.1 (2), 165.6, 174.2. 1MSIMS m/z 480.1779 [M⁺+H], calcd for C₂₅H₂₅N₃O₇ 480.1765.

1-(3-(3,4,5-trimethoxyphenyl)-3-(5-nitro-1H-indol-3-yl)propanoyl)-4-(5-nitro-1H-indol-3-yl)piperidin-2-one (3i): as a pale yellow semi liquid; IR (KBr) νmax: 656, 770, 819, 902, 1002, 1124, 1177, 1223, 1330, 1384, 1424, 1463, 1512, 1589, 1624, 1687, 2852, 2924 and 3342 cm⁻¹. ¹H NMR (300 MHz, CDCl₃): δ ppm 1.95-2.08 (1H, m), 2.19-2.32 (1H, m), 2.63-2.81 (1H, m), 2.93-2.05 (1H, m), 3.50-3.60 (2H, m), 3.61-3.68 (2H, m), 3.79 (6H, s), 3.84 (3H, s), 4.02-4.15 (1H, m), 4.89 (1H, m), 6.58 (1H, s), 6.59 (1H, s), 6.76 (1H, br d, J=2.2 Hz), 7.32-7.46 (3H, m), 8.07-8.17 (2H, m), 8.46-8.52 (3H, m), 8.69 (NH, brs). ¹³C NMR (75 MHz, CDCl₃): δ 28.5, 29.5, 39.8, 41.2, 42.9, 45.3, 56.2 (2), 60.7, 103.5, 105.0 (2), 110.3, 110.7, 110.9, 111.7, 112.1, 117.8, 118.1, 119.0, 121.8, 123.1, 126.3, 127.1, 133.0, 136.3, 139.5, 153.0 (2), 156.1, 159.2, 173.1, 175.5. HRESIMS m/z 664.2024 [M⁺+Na], calcd for C₃₃H₃₁N₅O₉ 664.2014.

1-(3-(5-methoxy-1H-indol-3-yl)-3-(3,4,5-trimethoxyphenyl)propanoyl)-5,6-dihydropyridin-2(M)-one (2j): as a pale yellow semi liquid; IR (KBr) νmax: 668, 768, 820, 1034, 1123, 1216, 1284, 1458, 1586, 1683, 2853, 2923 and 3468 cm⁻¹. ¹H NMR (300 MHz, CDCl₃): δ ppm 2.10-2.20 (2H, m), 3.67-3.77 (2H, m), 3.77-3.81 (12H, br s), 3.84-3.91 (2H, m), 4.79 (1H, t, J=7.5 Hz), 5.92-5.98 (1H, td, J=9.6, 1.7 Hz), 6.59 (2H, s), 6.81 (1H, m), 6.83 (1H, d, J=2.26 Hz), 6.98 (1H, d, J=2.26 Hz), 7.03 (1H, d, 1.7 Hz), 7.19-7.24 (1H, d, J=8.4 Hz), 7.93 (NH, br s). ¹³C NMR (75 MHz, CDCl₃): δ 24.5, 39.8, 41.3, 44.9, 55.8, 56.0 (2), 60.8, 101.4, 105.0 (2), 111.6, 112.2, 118.7, 122.2, 125.6, 127.1, 131.6, 136.2, 139.7, 145.5, 152.8 (2), 153.9, 165.6, 175.5. HRESIMS m/z 465.2042 [M⁺+H], calcd for C₂₆H₂₈N₂O₆ 465.2020.

1-(3-(3,4,5-trimethoxyphenyl)-3-(5-methyl-1H-indol-3-yl)propanoyl)-4-(5-methoxy-1H-indol-3-yl)piperidin-2-one (3j): as a pale yellow semi liquid; IR (KBr) νmax: 765, 1123, 1216, 1459, 1638, 2851, 2923 and 3438 cm⁻¹. ¹H NMR (300 MHz, CDCl₃): δ ppm 1.78-1.89 (1H, m), 2.09-2.19 (1H, m), 2.58-2.70 (1H, m), 2.87-2.97 (1H, dd, J=17.3, 5.2, Hz), 3.30-3.42 (1H, m), 3.45-3.58 (1H, m), 3.60-3.73 (2H, m), 3.78 (6H, s), 3.80 (3H, s), 3.81 (3H, s), 3.86 (3H, s), 3.87-3.98 (1H, m), 4.79 (1H, t, J=8.1 Hz), 6.60 (1H, s), 6.61 (1H, s), 6.69 (1H, br d, J=2.4 Hz), 6.81-6.87 (12H, m), 6.95-7.00 (2H, dd, J=7.5, 2.2 Hz), 7.10 (1H, dd, J=2.2 Hz), 7.21-7.29 (2H, m), 7.95 (2NH, br s). ¹³C NMR (75 MHz, CDCl₃): δ 28.7, 29.5, 40.0, 41.2, 43.0, 45.3, 55.8, 56.0, 56.2 (2), 60.8, 100.9, 101.6, 105.1 (2), 111.7, 112.0, 112.4, 115.9, 117.7, 118.5, 120.8, 122.3, 126.4, 127.2, 127.4, 131.8, 139.9, 149.9, 152.9 (2), 153.9, 154.0, 173.3, 175.8. HRESIMS m/z 612.2680 [M⁺+H], calcd for C₃₅H₃₇N₃O₇ 612.2704.

1-(3-(3,4,5-trimethoxyphenyl)-3-(5-methyl-1H-indol-3-yl)propanoyl)-5,6-dihydropyridin-2(1H)-one (2k): as a pale yellow semi liquid; IR (KBr) νmax: 594, 647, 675, 814, 847, 908, 1000, 1025, 1134, 1180, 1233, 1302, 1328, 1360, 1388, 1410, 1464, 1505, 1589, 1625, 1685, 2362, 2829, 2928, 2996, 3053, 3114, and 3385 cm⁻¹. ¹H NMR (300 MHz, CDCl₃): δ ppm 2.10-2.20 (2H, m), 2.41 (3H, s), 3.64-3.77 (2H, m), 3.79 (9H, s), 3.81-3.93 (2H, m), 4.81 (1H, t, J=7.5 Hz), 5.91-5.99 (1H, d, J=9.6 Hz), 6.59 (2H, s), 6.79-6.87 (1H, m), 7.01 (2H, br s,), 7.22 (1H, d, J=8.3 Hz), 7.34 (1H, s), 7.94 (NH, br s), ¹³C NMR (75 MHz, CDCl₃): δ 24.5, 29.6, 39.6, 41.2, 45.0, 56.0 (2), 60.7, 104.9 (2), 110.7, 118.5, 118.9, 121.5, 123.7, 125.6, 126.9, 128.5, 134.7, 136.2, 139.8, 145.4, 152.9 (2), 165.4, 175.0. HRESIMS m/z 487.1634 [M⁺+K], calcd for C₂₆H₂₈N₂O₅ 487.1630.

1-(3-(3,4,5-trimethoxyphenyl)-3-(5-methyl-1H-indol-3-yl)propanoyl)-4-(5-methyl-1H-indol-3-yl)piperidin-2-one (3k): as a pale yellow semi liquid; IR (KBr) νmax: 771, 1125, 1219, 1419, 1457, 1507, 1636, 2850, 2921, 2996, 3053, 3114, and 3457 cm⁻¹. ¹H NMR (300 MHz, CDCl₃): δ ppm 1.76-1.93 (1H, m), 2.00-2.10 (1H, m), 2.41 (3H, s), 2.45 (3H, s), 2.58-2.71 (1H, m), 2.87-2.97 (1H, dd, J=17.3, 5.2 Hz), 3.31-3.40 (1H, m), 3.46-3.60 (1H, m), 3.60-3.75 (2H, m), 3.78 (3H, s), 3.81 (6H, s), 3.84-3.98 (1H, m), 4.82 (1H, t, J=7.5 Hz), 6.60 (1H, s), 6.61 (1H, s), 6.80 (1H, br s), 6.96-7.10 (3H, m), 7.20-7,25 (2H, m), 7.28-7.37 (2H, m), 7.91 (NH, br s), 7.96 (NH, br s). ¹³C NMR (75 MHz, CDCl₃): δ 21.5, 28,7, 29.6, 39.7, 41.2, 43.1, 45,4, 49.4, 56.1 (2), 60.7, 105.0 (2), 110.7, 117.2, 117.6, 118.4, 118.9, 119.1, 120.1, 120.3, 121.5, 121.6, 123.4, 123.7, 124.0, 126.2, 128.6, 128.8, 134.8 (2), 140.0, 152.9 (2), 165.6, 175.8, HRESIMS m/z 618.2381 [M⁺+K], calcd for C₃₅H₃₇N₃O₅ 618.2365

### Example 3

Aldose Reductase Inhibition Studies

(I) Expression and Purification of Human Recombinant Aldose Reductase

Aldose reductase was cloned from human placenta in PMON 5997 plasmids, which were transformed into E.coli JM101 strain. Transformed cells were selected on LB-medium containing 50 μg/ml spectinomycin and were grown overnight at 37° C. in LB broth containing M9 medium supplemented with 1% casamino acids, 5 pg/ml thiamine, and 0.05% trace metals. Culture was induced by isopropyl thiogalactoside (IPTG) at the final concentration of 1 mM and grown for additional 2 hours. Cells were harvested by spinning at 2000 g for 5 min at 4° C. and subjected to osmotic fractionation by suspending them in 20% sucrose, 30 mM Tris pH 7.5, 1 mM EDTA, and then cells were incubated at 23° C. for 15 min. After incubation, cells were recovered by centrifugation for 15 min at 2000 g at 4° C. Supernatant (sucrose wash) was reserved and pellet was resuspended in 1 ml of ice cold deionized water and incubated for 10 min on ice. Again cells were recovered by centrifugation at 12000 g for 5 min at 4° C. Supernatant (water wash) was reserved and pellet was resuspended in 1 ml of ice cold deionized water. Osmotic shock extract was further subjected to purification.

Osmotic shock extract was subjected to 50-80% ammonium sulphate fractionation followed by centrifugation at 10,000 g for 20 min. The pellet obtained was resuspended in 100-200 ml of 25 mM imidazole-HCl, pH 7.4. Crude lysate was applied to chromatography column packed with PBE 94 chromatofocussing resin, which had been previously equilibrated with 25 mM imidazole. Proteins were eluted from the column with 1:8 diluted polybuffer pH 7.4. Column eluant was continuously monitored by measuring absorbance at 280 nm. Fractions containing ALR2 activity were pooled and dialyzed against 10 mM potassium phosphate buffer containing 0.5 mM EDTA. Dialyzed samples were then applied to 30×2.5 cm at hydroxylapatite column equilibrated with 10 mM potassium phosphate buffer pH 7.4 containing 0.5 mM EDTA at a flow rate of 60 ml/h. The enzyme was eluted with a linear gradient 10-300 mM potassium phosphate buffer, pH 7.0. Fractions from each purification step are subjected to SDS-PAGE. Fractions containing ALR2 activity were pooled, concentrated and stored at −20° C. until further inhibition studies.

(II) ALR2 Assay

The assay mixture in 1 ml contained 50 mM potassium phosphate buffer, pH 6.2, 0.2 mM lithium sulfate, 5 mM 2-mercaptoethanol, 1 mM DL-glyceraldehyde, 0.1 mM NADPH and recombinant ALR2. Appropriate blanks were employed for corrections. The assay mixture was incubated at 37° C. and initiated by the addition of NADPH at 37° C. The change in the absorbance at 340 nm due to NADPH oxidation was monitored in a Lamda35 spectrophotometer (Perkin-Elmer, Shelton, USA).

(III) ALR2 Inhibition and Determination of IC₅₀ Values

For inhibition studies concentrated stocks of compounds prepared in dimethyl sulfoxide were used and the final concentration of DMSO was not more than 1% of the assay volume. Various concentrations of the above mentioned analogues were added to assay mixture of ALR2 and incubated for 5 min before initiating the reaction by NADPH as described above. The percentage inhibition was calculated considering the activity in the absence of compound as 100%. The IC₅₀ values were determined by nonlinear regression analysis of the plot of percent inhibition versus log compound concentration.

(IV) Inhibition of Sorbitol Formation Under High Glucose Conditions by 3C, 3E and 2J in Ex Vivo System

In vitro incubation of RBC: Five mL blood was collected from healthy male volunteers on overnight fasting in heparinized tubes. Red blood cells were separated by centrifugation (3000 rpm/min about 30 min) and washed three times with isotonic saline at 4° C. Washed RBC were suspended in Kreb's-ringer bicarbonate buffer, pH 7.4 (pre-equilibrated with 5% CO₂) and incubated at 37° C. in presence of 5% CO₂ for 3 hrs under normal (5.5 mM) and high glucose (55 mM) conditions in duplicates. The effect of compounds on sorbitol accumulation was evaluated by incubating the RBC with different concentrations of compounds.

Estimation of sorbitol in RBC: At the end of incubation period, RBC was homogenized in 9 volumes of 0.8 M perchloric acid. The homogenate was centrifuged at 5,000 g at 4° C. for 10 min and the pH of the supernatant was adjusted to 3.5 with 0.5 M potassium carbonate. The sorbitol content of the supernatant was measured by fluorometric method using a spectrofluorometer (Jasco-FP-6500). Results of % inhibition in human RBC incubated under high glucose condition for compound 3c showed 58.5% inhibited the sorbitol formation, for compound 3e showed 68%, for compound 2j showed 64.9% respectively.

### Example 4

Molecular Docking Studies

Molecular docking studies were done by SYBYL FlexX software (Tripos). Ligand structures were constructed and minimized using the SYBYL modeling program. The FlexX module in SYBYL 7.0 was used to dock the compounds into the active site of the crystallographic structures, which was defined as all residues within 6.5 A° away from the inhibitor in original complex by using an incremental construction algorithm. For docking studies coordinates of crystal structure of protein (ALR2: PDB #1PWM) was taken from Brookhaven Protein Data Bank (PDB). The predicted protein ligand complexes were optimized and ranked according to the empirical scoring function ScreenScore, which estimates the binding free energy of the ligand receptor complex.

Results

TABLE 4
IC-50 values for selected Michael adducts.

| SL. NO | Compound/Standard | IC-50 (μM) |
|---|---|---|
| 1 | 3c | 4 |
| 2 | 3d | 40 |
| 3 | 3e | 4 |
| 4 | 2g | 15 |
| 5 | 2j | 8 |
| 6 | Piplartine | 160 |
| 7 | Quercetin | 40 |
| 8 | Sorbinil | 8 |

Data are average of four experimental values (refer to FIG. 8)

### Advantages of the Invention

ALR2 mediated sorbitol formation leads to various diabetic complications (cataract, retinopathy, neuropathy and nephropathy). Therefore, ALR2 is a drug target for diabetic complications. Largely, two chemical classes of ALR2 inhibitors (ARI) have been tested in phase III clinical trials. While carboxylic acid inhibitors (zopolrestat, ponalrestat and tolrestat) have shown poor tissue permeability and are not very potent in vivo, and the other class of ARIs, spirohydantoin inhibitors (sorbinil, fidarestat) penetrate tissues more efficiently, showing better pharmacokinetics but incomplete enzyme inhibition, and associated with skin hypersensitivity reactions and liver toxicity. Thus, there is a need for developing and evaluating newer ARIs considering efficacy and safety issues. To overcome these limitations, using the natural compounds piplartine (isolated from Piper chaba) as a lead compound, various analogues were synthesized via Micheal addition to inhibit ALR2. These compounds have inhibited ALR2 in vitro and as well as sorbitol accumulation in ex vivo. Molecular docking studies indicates that there compounds not only binds to active site but also extended into the hydrophobic pocket and this might impart specificity of inhibition of ALR2 over other related reductases. Therefore,this invention has led to development of selective and potent ALR2 inhibitors to prevent diabetic complications.

1. A compound of general formula A 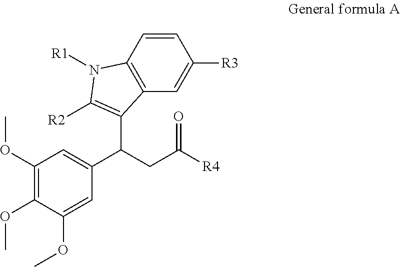
wherein R₁=Hyrogen, Methyl or Benzyl;
R₂=Methyl or Phenyl;
R₃=Nitro, Fluro, Bromo, Iodo, Methyl or Methoxy;
R4=

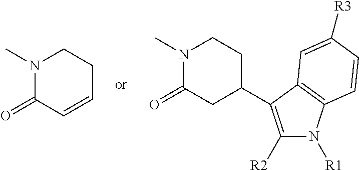

2. The compound as claimed in claim 1, wherein representative compounds of general formula A are: 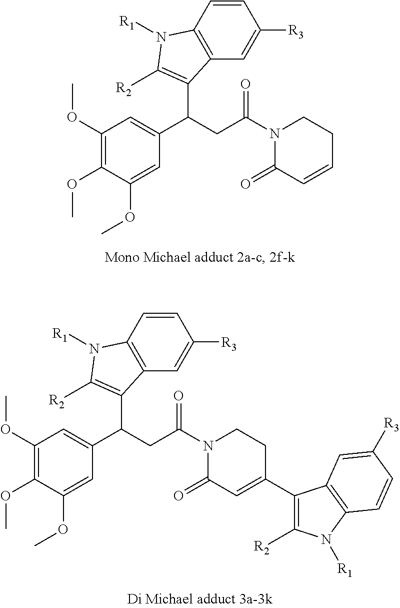
Wherein R₁=Hyrogen, Methyl or Benzyl;
R₂=Methyl or Phenyl and
R₃=Nitro, Fluro, Bromo, Iodo, Methyl or Methoxy.

3. The compound as claimed in claim 1, wherein representative compounds of general formula A comprising:
[1-(3-(1H-indol-3-yl)-3-(3,4,5-trimethoxyphenyl)propanoyl)-5,6-dihydropyridin-2(1H)-one] (2a);
[1-(3-(1H-indol-3-yl)-3-(3,4,5-trimethoxyphenyl)propanoyl)-4-(1H-indol-3-yl)piperidin-2-one] (3a);
[1-(3-(3,4,5-trimethoxyphenyl)-3-(1-methyl-1H-indol-3-yl)propanoyl)-5,6-dihydropyridin-2(1H)-one] (2b);
[1-(3-(3,4,5-trimethoxyphenyl)-3-(1-methyl-1H-indol-3-yl)propanoyl)-4-(1-methyl-1H-indol-3-yl)piperidin-2-one] (3b);
[1-(3-(1-benzyl-1H-indol-3-yl)-3-(3,4,5-trimethoxyphenyl)propanoyl)-5,6-dihydropyridin-2(1H)-one] (2c);
[1-(3-(1benzyl-1H-indol-3-yl)-3-(3,4,5-trimethoxyphenyl)propanoyl)-4-(1-benzyl-1H-indol-3-yl)piperidin-2-one] (3c);
[1-(3-(3,4,5-trimethoxyphenyl)-3-(2-methyl-1H-indol-3-yl)propanoyl)-4-(2-methyl-1H-indol-3-yl)piperidin-2-one] (3d);
[1-(3-(3,4,5-trimethoxyphenyl)-3-(2-phenyl-1H-indol-3-yl)propanoyl)-4-(2-phenyl-1H-indol-3-yl)piperidin-2-one] (3e);
[1-(3-(5-iodo-1H-indol-3-yl)-3-(3,4,5-trimethoxyphenyl)propanoyl)-5,6-dihydropyridin-2(1H)-one] (2f);
[1-(3-(5-iodo-1H-indol-3-yl)-3-(3,4,5-trimethoxyphenyl)propanoyl)-4-(5-iodo-1H-indol-3-yl)piperidin-2-one] (3f);
[1-(3-(5-bromo-1H-indol-3-yl)-3-(3,4,5-trimethoxyphenyl)propanoyl)-5,6-dihydropyridin-2(1H)-one] (2g);
[1-(3-(5-bromo-1H-indol-3-yl)-3-(3,4,5-trimethoxyphenyl)propanoyl)-4-(5-bromo-1H-indol-3-yl)piperidin-2-one] (3g);
[1-(3-(5-fluoro-1H-indol-3-yl)-3-(3,4,5-trimethoxyphenyl)propanoyl)-5,6-dihydropyridin-2(1H)-one] (2h);
[1-(3-(5-fluoro-1H-indol-3-yl)-3-(3,4,5-trimethoxyphenyl)propanoyl)-4-(5-fluoro-1H-indol-3-yl)piperidin-2-one] (3h);
[1-(3-(3,4,5-trimethoxyphenyl)-3-(5-nitro-1H-indol-3-yl)propanoyl)-5,6-dihydropyridin-2(1H)-one] (2i);
[-(3-(3,4,5-trimethoxyphenyl)-3-(5-nitro-1H-indol-3-yl)propanoyl)-4-(5-nitro-1H-indol-3-yl)piperidin-2-one] (3i);
[-(3-(5-methoxy-1H-indol-3-yl)-3-(3,4,5-trimethoxyphenyl)propanoyl)-5,6-dihydropyridin-2(1H)-one] (2j);
[(5-MethoxyIndoleDs)1-(3-(3,4,5-trimethoxyphenyl)-3-(5-methyl-1H-indol-3-yl)propanoyl)-4-(5-methoxy-1H-indol-3-yl)piperidin-2-one] (3j);
[(5-MethylIndoleMs)1-(3-(3,4,5-trimethoxyphenyl)-3-(5-methyl-1H-indol-3-yl)propanoyl)-5,6-dihydropyridin-2(1H)-one] (2k);
[(5-MethylIndoleDs)1-(3-(3,4,5-trimethoxyphenyl)-3-(5-methyl-1H-indol-3-yl)propanoyl)-4-(5-methyl-1H-indol-3-yl)piperidin-2-one] (3k).

4. The compound as claimed in claim 1, wherein structural formula of the representative compounds of general formula A comprising:

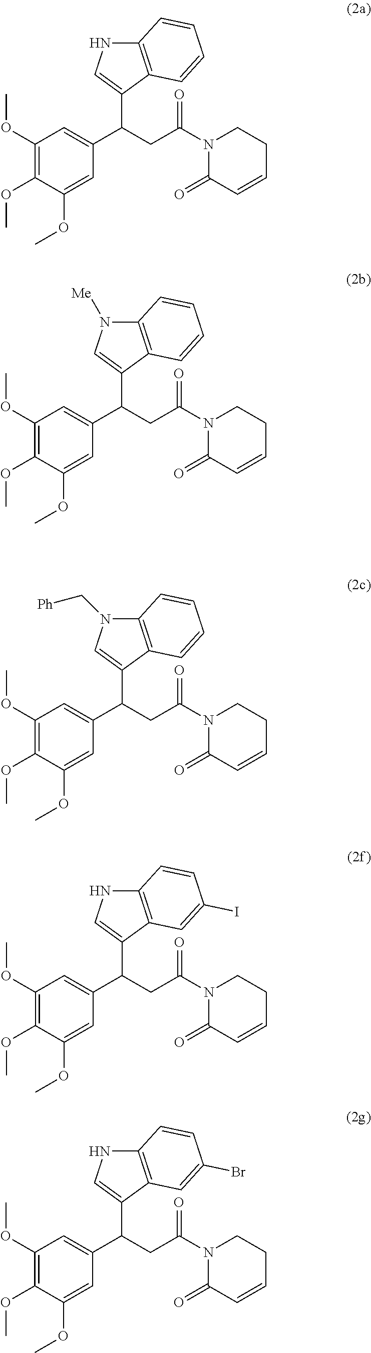

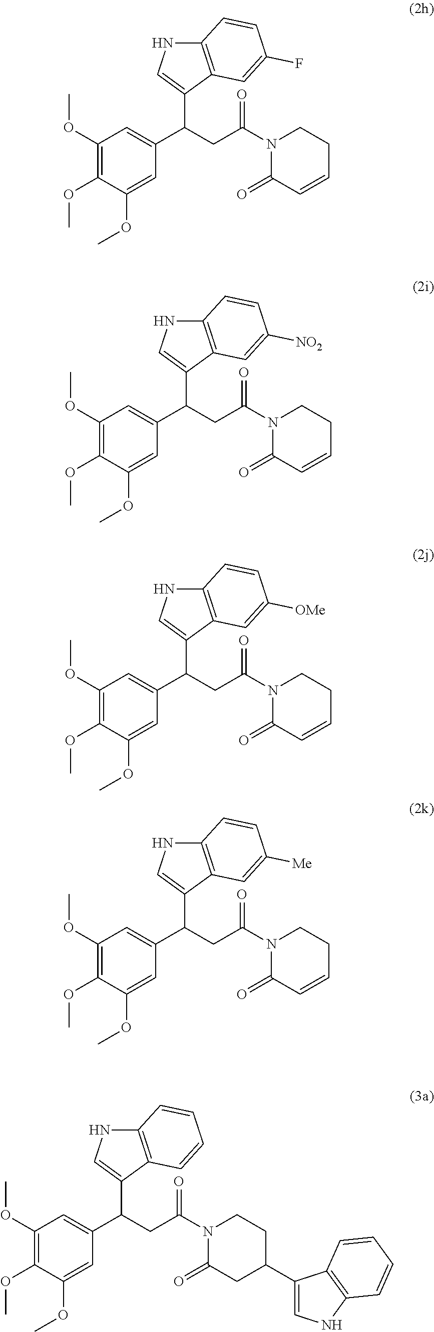

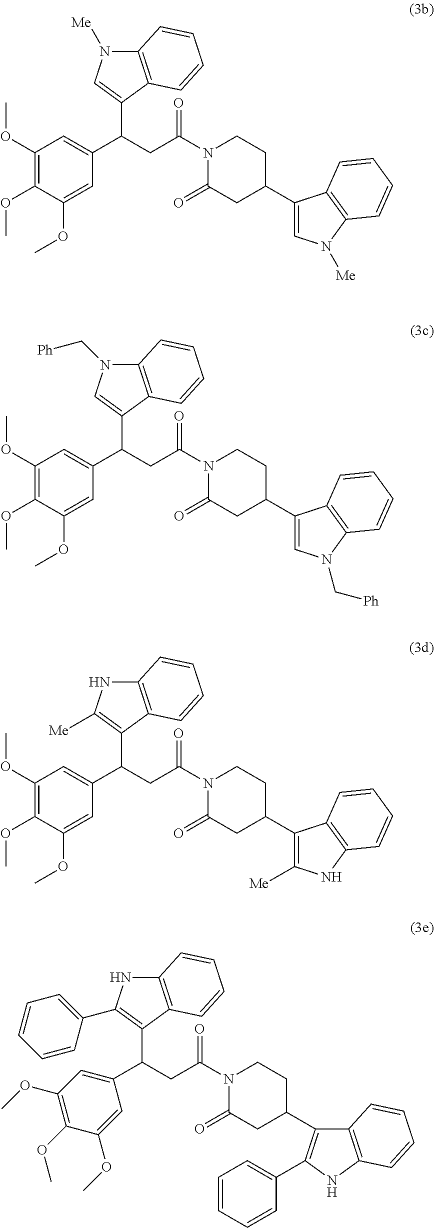

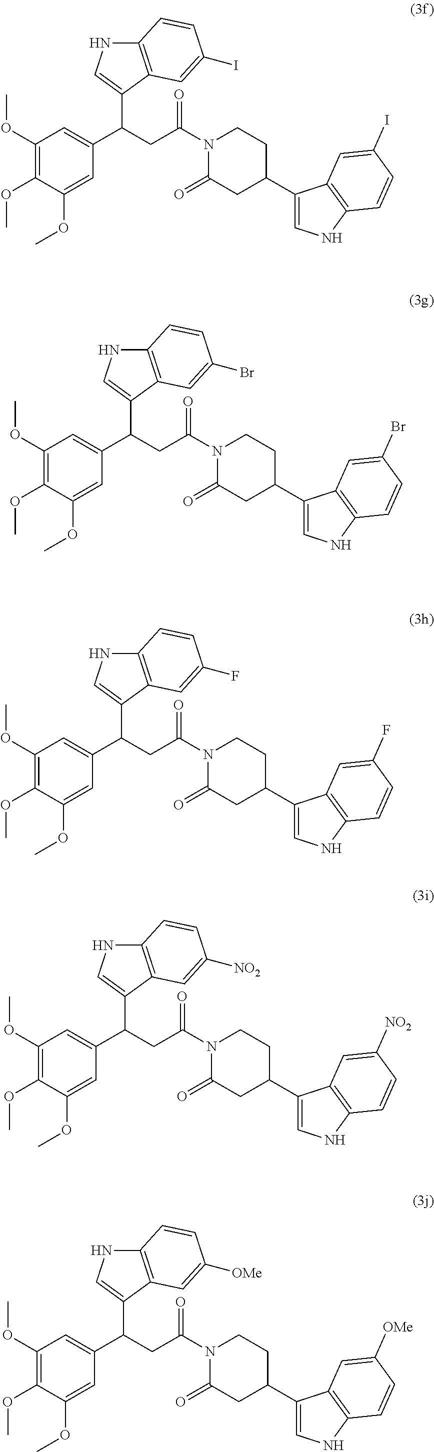

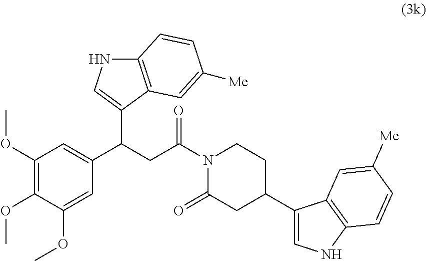

5. A compound of general formula A are useful for anti-diabetic complications having (Aldose reductase) ALR2 inhibitory activity.

6. A process for the preparation of compound of general formula A by Michael addition and the said process comprising the steps of:
i. mixing piplartine and substituted indole In the ratio ranging between 1:3 to 1:5 with the catalyst in the ratio ranging between 10 to 12 mole % to obtain a mixture;
ii. refluxing the mixture as obtained in step (i) in solvent at temperature in the range of 60-100° C. for a period in the range of 12 to 48 h till complete conversion;
evaporating the solvent of the refluxed product as obtained in step (ii) followed by washing with saturated hypo solution followed by extraction with chloroform to obtain combined organic layer;
iv. drying the combined organic layer as obtained in step (iii) over anhydrous sodium sulphate followed by evaporating using rotary evaporator to obtain the product;
v. purifying the product as obtained in step (iv) by silica-gel column chromatography to obtain pure product of general formula A.

7. The process as claimed in step (i) of claim 6, wherein substituted indole used is selected from the group consisting of Indole, 2-methylindole, 1-benzyllindole, 2-methylindole, 2-phenylindole, 5-iodoindole,5-bromoindole, 5-fluoroindole, 5-nitroindole, 5-methoxyindole or 5-methylindole.

8. The process as claimed in step (i) of claim 6, wherein catalyst used is iodine.

9. The process as claimed in step (ii) of claim 6, wherein solvent used is selected from the group consisting of 1,2-Dichloroethane, Dichloromethane, Methanol and Acetonitrile preferably 1,2-Dichloroethane.

10. The compounds as claimed in claim 3, wherein compound 3c and 3e exhibiting highest ALR2 inhibition with IC₅₀ values 4 μM.

11. The compounds as claimed in claim 3, wherein compound 2j and 2g exhibiting ALR2 inhibition with IC₅₀ values 8 and 15 μM respectively.

12. The compounds as claimed in claim 3, wherein the said compounds are effective in inhibiting human ALR2 in vitro wherein the said compounds are useful treating the diabetic complications in mammals upon administration of compounds.

13. The compounds as claimed in claim 3, wherein the said compounds as ALR2 inhibitors is supported by molecular docking data, wherein the said compounds 3c, 3e and 2j bind to ALR2 making contacts with active site residues ALA299, LEU300, SER302.

14. The compounds as claimed in claim 1, wherein the said compounds are effective in suppressing the formation of sorbitol in RBC under high glucose conditions ex vivo.

15. The compounds as claimed in claim 1, wherein the said compounds comprise potential against diabetic complications like diabetic cataract, diabetic nephropathy, diabetic neuropathy, diabetic corneal keratopahty, diabetic retinopathy, diabetic dermopathty and other diabetic microangeopathics.

16. The compounds as claimed in claim 1, wherein the said compounds inhibit epithelial to mesenchymal transition in diabetic retinopathy.

17. The compounds as claimed in claim 1, wherein the said compounds are used as a prodrug and pharmacological carriers to inhibit diabetic complications like diabetic cataract and diabetic retinopathy.
